SEQUENCE LISTING (78 sequences)

SEQ ID NO: 1 - DNA, 25 bp, Artificial Sequence
  PCR primers
gttcaaagaggtcgtgctcagagaa

SEQ ID NO: 2 - DNA, 25 bp, Artificial Sequence
  PCR primers
gttcaaagagtggtagctcagagaa

SEQ ID NO: 3 - DNA, 25 bp, Artificial Sequence
  PCR primers
gttcaaagagttcctgctcagagaa

SEQ ID NO: 4 - RNA, 107 nt, Artificial Sequence
  Stem-loop Structure of RNA I
acaguauuugguaucugcgcucugcugaagccaguuaccuucggaaaaagaguugguagcucuugauccggcaaacaaaccaccguugguagcggugguuuuuuugu

SEQ ID NO: 5 - RNA, 103 nt, Artificial Sequence
  Stem-loop Structure of RNA I
acaaguuuuggugacugcgcuccuccaagccaguuaccuggguucaaagaguugguagcucagagaaccuucgaaaaaccgcccugcaaggcgguuuuuucgu

SEQ ID NO: 6 - RNA, 105 nt, Artificial Sequence
  Stem-loop Structure of RNA I
acagauuuuggugacugcgcuccuccaagccaguuaccuugguucaaagaguugguagcucagcgaaccuugagaaaaccaccguugguagcggugguuuuucuu

SEQ ID NO: 7 - RNA, 104 nt, Artificial Sequence
  Stem-loop Structure of RNA I
acagauuugguugcugugcucugcgaaagccaguuaccacgguuaagcaguuccccaacugacuuaaccuucgaucaaaccaccuccccaggugguuuuuucgu

SEQ ID NO: 8 - DNA, 30 bp, Artificial Sequence
  PCR primers
ctctttgaaccgaggtaactggcttggagg

SEQ ID NO: 9 - DNA, 47 bp, Artificial Sequence
  PCR primers
caagccagttacctcggttcaaagagtnnnnngctcagagaaccttc

SEQ ID NO: 10 - DNA, 22 bp, Artificial Sequence
  PCR primers
ggcgagccagcctgtggggttt

SEQ ID NO: 11 - DNA, 24 bp, Artificial Sequence
  PCR primers
ggcgaggctatttattgcccgttg

SEQ ID NO: 12 - DNA, 26 bp, Artificial Sequence
  PCR primers
ggcgacactcgtctgcgggtacagta

SEQ ID NO: 13 - DNA, 24 bp, Artificial Sequence
  PCR primers
ggcgttacgctgattgacaatcgg

SEQ ID NO: 14 - DNA, 27 bp, Artificial Sequence
  PCR primers
ggcgacacgcaaacacaacaataacgg

SEQ ID NO: 15 - DNA, 26 bp, Artificial Sequence
  PCR primers
ggcgttaccagcgtggaatatcagtc

SEQ ID NO: 16 - DNA, 38 bp, Artificial Sequence
  PCR primers
agttattggtgcccttacccactgacaatccgtaaaga

SEQ ID NO: 17 - DNA, 34 bp, Artificial Sequence
  PCR primers
tagcaccaggcgtttcagggtgcaagacgatcaa

SEQ ID NO: 18 - DNA, 36 bp, Artificial Sequence
  PCR primers
gttattggtgcccttagccagcgattatcaaaacaa

SEQ ID NO: 19 - DNA, 32 bp, Artificial Sequence
  PCR primers
tagcaccaggcgttcggcgaaagtcatcagga

SEQ ID NO: 20 - DNA, 37 bp, Artificial Sequence
  PCR primers
agttattggtgcccttaggtgcccatcatcaagaagg

SEQ ID NO: 21 - DNA, 35 bp, Artificial Sequence
  PCR primers
tagcaccaggcgttcggctgttggaatcactcatc

SEQ ID NO: 22 - DNA, 36 bp, Artificial Sequence
  PCR primers
agttattggtgcccttagcggaaacgcagtctctta

SEQ ID NO: 23 - DNA, 34 bp, Artificial Sequence
  PCR primers
tagcaccaggcgtttgaaattcaatgccggaaag

SEQ ID NO: 24 - DNA, 41 bp, Artificial Sequence
  PCR primers
agttattggtgcccttaaccagacataagaaggtgaatagc

SEQ ID NO: 25 - DNA, 37 bp, Artificial Sequence
  PCR primers
tagcaccaggcgttacccttaaataagagactgcgtt

SEQ ID NO: 26 - DNA, 39 bp, Artificial Sequence
  PCR primers
agttattggtgcccttaggcattgaatttcaaaataagg

SEQ ID NO: 27 - DNA, 31 bp, Artificial Sequence
  PCR primers
tagcaccaggcgttaaaagcccggcgataag

SEQ ID NO: 28 - DNA, 36 bp, Artificial Sequence
  PCR primers
ccacaggctggctcgccagagtcccgctcagaagaa

SEQ ID NO: 29 - DNA, 37 bp, Artificial Sequence
  PCR primers
ggcaataaatagcctcgccttcgaaatgaccgaccaa

SEQ ID NO: 30 - DNA, 39 bp, Artificial Sequence
  PCR primers
cccgcagacgagtgtcgcctaagggcaccaataactgcc

SEQ ID NO: 31 - DNA, 36 bp, Artificial Sequence
  PCR primers
tgtcaatcagcgtaacgccaacgcctggtgctacgc

SEQ ID NO: 32 - DNA, 41 bp, Artificial Sequence
  PCR primers
attccacgctggtaacgccttcaccgacaaacaacagataa

SEQ ID NO: 33 - DNA, 40 bp, Artificial Sequence
  PCR primers
gttgtgtttgcgtgtcgccgtgcttagtgcatctaacgct

SEQ ID NO: 34 - DNA, 20 bp, Artificial Sequence
  PCR primers
ctggagtgaataccacgacg

SEQ ID NO: 35 - DNA, 19 bp, Artificial Sequence
  PCR primers
ggattggctgagacgaaaa

SEQ ID NO: 36 - DNA, 19 bp, Artificial Sequence
  PCR primers
gtccggtgccctgaatgaa

SEQ ID NO: 37 - DNA, 20 bp, Artificial Sequence
  PCR primers
cccaatagcagccagtccct

SEQ ID NO: 38 - DNA, 22 bp, Artificial Sequence
  PCR primers
cgctcacgcaactggtccagaa

SEQ ID NO: 39 - DNA, 23 bp, Artificial Sequence
  PCR primers
cgaggcatagactgtaccccaaa

SEQ ID NO: 40 - DNA, 20 bp, Artificial Sequence
  PCR primers
ttgtcggcggtggtgatgtc

SEQ ID NO: 41 - DNA, 24 bp, Artificial Sequence
  PCR primers
atgcggtgaactgtggaataaacg

SEQ ID NO: 42 - DNA, 19 bp, Artificial Sequence
  PCR primers
ggtagagacgccattgtag

SEQ ID NO: 43 - DNA, 18 bp, Artificial Sequence
  PCR primers
ccgatccacatagcaaca

SEQ ID NO: 44 - DNA, 20 bp, Artificial Sequence
  PCR primers
ccgtatcgtagaatgctatt

SEQ ID NO: 45 - DNA, 18 bp, Artificial Sequence
  PCR primers
ccgctttggtgaagaata

SEQ ID NO: 46 - DNA, 23 bp, Artificial Sequence
  PCR primers
gcgttgagaacctacctgctaat

SEQ ID NO: 47 - DNA, 21 bp, Artificial Sequence
  PCR primers
atcctcggtgatggcattgaa

SEQ ID NO: 48 - DNA, 70 bp, Artificial Sequence
  PCR primers
gttgtagagagttgagttcatggaatccctgacgttacaacccatcgctcgtgtaggctggagctgcttc

SEQ ID NO: 49 - DNA, 70 bp, Artificial Sequence
  PCR primers
cattcaggctgcctggctaatccgcgccagctgctcgaaataatccggagcatatgaatatcctccttag

SEQ ID NO: 50 - DNA, 69 bp, Artificial Sequence
  PCR primers
ctgcgggtacagtaattaaggtggatgtcgcgttatggagaggattgtcgtgtaggctggagctgcttc

SEQ ID NO: 51 - DNA, 70 bp, Artificial Sequence
  PCR primers
ccccatttcagcttcaatggcatgaccaaaggtgtgtcccagattcagagcatatgaatatcctccttag

SEQ ID NO: 52 - DNA, 69 bp, Artificial Sequence
  PCR primers
cggagccgtgatggctggaaacacaattggacaactctttcgcgtaaccgtgtaggctggagctgcttc

SEQ ID NO: 53 - DNA, 70 bp, Artificial Sequence
  PCR primers
ccagcgtggaatatcagtcttcacatcggcattttgcgcccgttgccgagcatatgaatatcctccttag

SEQ ID NO: 54 - DNA, 69 bp, Artificial Sequence
  PCR primers
aaacgcgaccgcatcgtcttgaataactgtcagttacaaaatccacagcgtgtaggctggagctgcttc

SEQ ID NO: 55 - DNA, 72 bp, Artificial Sequence
  PCR primers
tcagggtgcaagacgatcaatcttccacgcatcattttcacgctggtacgagcatatgaatatcctccttag

SEQ ID NO: 56 - DNA, 69 bp, Artificial Sequence
  PCR primers
tctattgtgtcgcgcttttgccttccggcatagttctgtttatgcttctgtgtaggctggagctgcttc

SEQ ID NO: 57 - DNA, 73 bp, Artificial Sequence
  PCR primers
catacacttcgtcatcgcttcgatcactgcagcacggaagcctttctcttgagcatatgaatatcctccttag

SEQ ID NO: 58 - DNA, 70 bp, Artificial Sequence
  PCR primers
tagaatgctcgccgtttacctgtttcgcgccacttccggtgcccatcatcgtgtaggctggagctgcttc

SEQ ID NO: 59 - DNA, 72 bp, Artificial Sequence
  PCR primers
gttaccgggcgaccaatcaccatataatcaacaccagccgacaacgcctgagcatatgaatatcctccttag

SEQ ID NO: 60 - DNA, 69 bp, Artificial Sequence
  PCR primers
gccgtaaggtgaatgttttacgtttaacctggcaaccagacataagaaggtgtaggctggagctgcttc

SEQ ID NO: 61 - DNA, 72 bp, Artificial Sequence
  PCR primers
ccctaaccgagcctgcgcaaaagcaatcgcctgcgccacctgctgcggtgagcatatgaatatcctccttag

SEQ ID NO: 62 - DNA, 23 bp, Artificial Sequence
  PCR primers
cgctgacagacttcatggttgag

SEQ ID NO: 63 - DNA, 22 bp, Artificial Sequence
  PCR primers
agggcaccttctgcgtgtaatg

SEQ ID NO: 64 - DNA, 18 bp, Artificial Sequence
  PCR primers
cggcacagccctgattgg

SEQ ID NO: 65 - DNA, 22 bp, Artificial Sequence
  PCR primers
aacgcaatccgctgtatgaaga

SEQ ID NO: 66 - DNA, 21 bp, Artificial Sequence
  PCR primers
cagaggagccagggtttcgtt

SEQ ID NO: 67 - DNA, 23 bp, Artificial Sequence
  PCR primers
ccgctgcgaacttcactcttacc

SEQ ID NO: 68 - DNA, 18 bp, Artificial Sequence
  PCR primers
taacggcggcgatggtgt

SEQ ID NO: 69 - DNA, 20 bp, Artificial Sequence
  PCR primers
acaagccaatgctggtgccg

SEQ ID NO: 70 - DNA, 24 bp, Artificial Sequence
  PCR primers
cctggctactcagacgctggataa

SEQ ID NO: 71 - DNA, 23 bp, Artificial Sequence
  PCR primers
acggaatctatgttttctggtcg

SEQ ID NO: 72 - DNA, 24 bp, Artificial Sequence
  PCR primers
ctttttcgtcgtaatgtttgagta

SEQ ID NO: 73 - DNA, 22 bp, Artificial Sequence
  PCR primers
catccacccaaattgtcataaa

SEQ ID NO: 74 - DNA, 18 bp, Artificial Sequence
  PCR primers
cgattctgcggcgttgat

SEQ ID NO: 75 - DNA, 18 bp, Artificial Sequence
  PCR primers
ctgccaggggagaaatcg

SEQ ID NO: 76 - DNA, 22 bp, Artificial Sequence
  PCR primers
gaactcctgaccgaatacctgt

SEQ ID NO: 77 - DNA, 23 bp, Artificial Sequence
  PCR primers
gtttttcattgttgacacacctc

SEQ ID NO: 78 - DNA, 23 bp, Artificial Sequence
  PCR primers
acccttaaataagagactgcgtt

(19) United States
(12) Patent Application Publication
(10) Pub. No.: US 2016/0304884 A1
(43) Pub. Date: Oct. 20, 2016

(54) Compositions and Methods for Expressing Nucleic Acid Sequences

(71) Applicant: NATIONAL UNIVERSITY OF SINGAPORE, Singapore (SG)

(72) Inventors: Heng Phon Too, Singapore (SG); Ruiyang Zou, Singapore (SG)

(21) Appl. No.: 15/101,207

(22) PCT Filed: Nov. 26, 2014
(86) PCT No.: PCT/SG2014/000556

§ 371 Date: Jun. 2, 2016

Related U.S. Application Data
(60) Provisional application No. 61/910,617, filed on Dec. 2, 2013.

(51) Int. Cl. C12N 15/70; C12N 15/10

(52) U.S. Cl. CPC C12N 15/70; C12N 15/1093

(57) ABSTRACT

Described herein is the development of a multi-plasmid system (Compatible Antibiotic-free Multi-Plasmid System, CAMPS) for the expression of one or more nucleic acid sequences of interest in specially engineered host cells and grown in antibiotic-free medium. A panel of compatible plasmids was engineered in which each plasmid comprises an identical origin of replication (Ori) which only differs between plasmids in the loop sequence/s of the RNA I/II region of the Ori. Thus, these plasmids share the same replication mechanism but vary in copy numbers. In order to maintain these multiple plasmids without using antibiotics, multiple conditional essential genes (CEG) from the host genome were grafted into these plasmids. As a result, all of these co-existing plasmids carrying the CEG were maintained in a host where the corresponding CEGs were knocked out from the host's genome during fermentation. Said CAMPS system has broad utility in metabolic engineering and synthetic biology.

### BACKGROUND OF THE INVENTION

In modern biotechnology, the fermentation of genetically modified microbes has been industrialized for the production of large biomolecules such as protein or DNA. In these instances, only a limited number of genetic modifications is introduced into the cells. With the recent developments in genomics, microbes are now used to produce valuable chemical compounds by metabolic engineering and to generate novel biological phenotypes by synthetic biology. These processes often involve the modifications of one or more intricate biological pathways which necessitate the manipulations of multiple genes.

In bacteria, plasmids are routinely used in metabolic engineering and synthetic biology simply because these are easily manipulated and can have multiple copies in cells. There are however, significant limitations restricting the utility of multiple plasmids in large scale fermentation processes. Firstly, the co-existence of two or more incompatible plasmids will invariably be regulated resulting in unstable copy numbers whereby some of these plasmids will be lost over prolong periods of fermentation. One approach to circumvent this is to use plasmids that are naturally compatible. However, there are only limited numbers of these plasmids with distinct copy numbers. These plasmids have different replication mechanisms which are known to respond differently to environmental cues making the simultaneous use of these plasmids a challenge. Secondly, because they are non-essential for growth, plasmids may be lost during the scaling-up process where cells are known to be metabolically stressed by the over-expression of genes carried by the plasmid. One approach to circumvent this is to use antibiotics to exert selection pressures. Unfortunately, the use of antibiotics is both costs prohibitive, unstable in long term growth and faces significant regulatory issues.

Hence, to be industrially relevant, combinatorial panels of plasmids which can stably co-exist in a cell and grow under selection pressure without the use of antibiotics are needed.

### SUMMARY OF THE INVENTION

Described herein is the development of a multi-plasmid system (Compatible Antibiotic-free Multi-Plasmid ystem, CAMPS) for the expression of one or more (multiple; a plurality) nucleic acid sequences of interest (e.g., genes of interest (GOI)) carried by a one or more (multiple; a plurality) multi-compatible plasmids in specially engineered host cells and grown in antibiotic-free medium.

Accordingly, in one aspect the invention is directed to a method of expressing a plurality (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.) of nucleic acid sequences comprising maintaining a (one or more) host cell comprising one or more, and preferably, two or more plasmids. In a particular aspect, the two or more plasmids are compatible. The host cell lacks one or more conditional essential genes, and the two or more plasmids comprise the two or more nucleic acid sequences to be expressed and the sequences of the one or more conditional essential genes, and each plasmid further comprises an origin of replication (Ori) that is identical except for one or more loop sequences in the Ori of each plasmid. The host cell is maintained under conditions in which the two or more nucleic acid sequences and the one or more conditional essential genes are expressed in the host cell, thereby expressing the two or more nucleic acid sequences.

In another aspect, the invention is directed to a host cell comprising two or more plasmids, wherein each plasmid comprises one or more conditional essential genes (CEGs). In another aspect, the invention is directed to a host cell wherein (i) the host cell lacks one or more conditional essential genes, and (ii) the two or more plasmids comprise the one or more conditional essential genes. The plasmids can further comprise an Ori that is identical except for one or more loop sequences in the Ori of each plasmid.

In yet another aspect, the invention is directed to a plurality of plasmids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 etc), such as a panel of plasmids. In one aspect, the plurality of plasmids comprises two or more plasmids.

In another system, the invention is directed to a system for expressing two or more nucleic acid sequences comprising a host cell and two or more plasmids, wherein the host cell lacks one or more conditional essential genes and the two or more plasmids comprise the one or more conditional essential genes, and each plasmid comprises an origin of replication (Ori) that is identical except for one or more loop sequences in the Ori of each plasmid.

In yet another embodiment, the invention is directed to a method of preparing a library of compatible plasmids comprising introducing into a host cell at least two plasmids wherein each plasmid comprises an origin of replication (Ori) that is identical except for one or more loop sequences in the Ori of each plasmid; and maintaining the host cell under conditions in which the plasmids are replicated in the host cell, thereby by preparing a library of compatible plasmids.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 Illustration of CAMPS.

FIG. 2 Design of compatible plasmids—Illustration of the mechanisms involved in the regulation of plasmid replication; the detailed description can be found in the main text. Original Plasmid section: the mechanism of the replication of p15A plasmid. Incompatible Plasmid section: the mechanism of incompatibility caused by the coexisting of plasmid with the same origin of replication. Compatible Plasmid section: the design of novel plasmids and the mechanism of their compatibility.

FIGS. 3A-3C Construction of compatible plasmid library (FIG. 3A) Illustration of the thiophosphate mediated site-directed mutagenesis used to engineer the 2nd loop of p15A Ori site; (FIG. 3B) the copy number of screened plasmids with artificial Ori sites in MG1655, DE3 strain measured by qPCR; The mutants' 2nd loop sequences were presented in X-axis and the original one was used as the control (sequence: TGGTA). Except the control, all the plasmids were ascendingly sorted according to their copy numbers. Two biological replicates were carried out for each condition and the standard errors were presented. (FIG. 3C) the concentrations of extracted plasmids; the same amount of cells were used for column based plasmid extraction and the same amount of water were used to elute the plasmid.

FIGS. 4A-4C Compatibility test for dual-plasmid system—(FIG. 4A) the 2nd loop sequences of three selected Ori sites (p15A (SEQ ID NO: 1), p15AL2-5 (SEQ ID NO: 2), p15AL2-8 (SEQ ID NO: 3)), the plasmids used in the study and their abbreviations; FIGS. 4B, 4C the copy numbers of the original (T7-ADS-ispA-Cam-p15A, T7-dxs-Kan-p15A) and artificial (T7-ADS-ispA-Cam-p15AL2-5, T7-ADS-ispA-Cam-p15AL2-8) plasmids at various standing along or coexisting conditions in MG1655, DE3 strain when all relevant antibiotics (FIG. 4B) or only selected antibiotics (FIG. 4C) were supplied to the medium. The copy numbers were measured by qPCR. Kan-plasmid: the plasmids carrying kanamycin (Kan) resistant gene. Can-plasmid: the plasmids carrying chloramphenicol (Cam) resistant gene. Three biological replicates were carried out for each condition and the standard errors were presented.

FIGS. 5A-5C Compatibility test for triple-plasmid system—(FIG. 5A), the 2nd loop sequences of three selected Ori sites (p15A (SEQ ID NO: 1), p15AL2-5 (SEQ ID NO: 2), p15AL2-8 (SEQ ID NO: 3)) and the two groups of plasmids used in the study (Original group and Engineered group); FIGS. 5B, 5C the copy numbers of the plasmids when all relevant antibiotics (FIG. 5B) or only selected antibiotics (FIG. 5C) were supplied to the medium. The plasmids were introduced into MG1655, DE3 strain either by groups (Original, Engineered) or separately (control) and measured by qPCR. The Kan-plasmid: the plasmids carrying kanamycin (Kan) resistant gene. Cam-plasmid: the plasmids carrying chloramphenicol (Cam) resistant gene. Spec-plasmid: the plasmids carrying spectinomycin (Spec) resistant gene. Three biological replicates were carried out for each condition and the standard errors were presented.

FIG. 6 Construction of antibiotic-free multi-plasmids/host system—Illustration of the workflow to construct a multi-plasmid system which can be stably maintained inside the engineered E. coli host without the usage of antibiotics.

FIGS. 7A-7C Plasmid stability test for various plasmid/host systems—(FIG. 7A) the plasmid copy numbers of single-plasmid/host systems measured by qPCR; The T7-dxs-Kan-aroA-pET, T7-ADS-Cam-aroB-p15A and T7-CYP450-CPR-Spec-aroC-pCL plasmids carrying conditional essential genes were introduced to either the native host (MG1655 (DE3) strain) or engineered host with relevant gene knockout. (FIG. 7B) the plasmid copy numbers of dual-plasmid/host system measured by qPCR; both T7-ADS-Cam-aroB-p15A and T7-CYP450-CPR-Spec-aroC-pCL plasmids were introduced to MG1655, DE3, AaroBC strain. (FIG. 7C) the plasmid copy numbers of triple-plasmid/host system measured by qPCR; T7-dxs-Kan-aroA-pET, T7-ADS-Cam-aroB-p15A and T7-CYP450-CPR-Spec-aroC-pCL plasmids were introduced to MG1655, DE3, ΔaroABC strain together. 0.1 mM of Isopropyl β-D-1-thiogalactopyranoside (IPTG) was supplied in certain conditions to induce the genes carried by the plasmids to generate selection stress. Kan-plasmid: the plasmids carrying kanamycin (Kan) resistant gene. Cam-plasmid: the plasmids carrying chloramphenicol (Cam) resistant gene. Spec-plasmid: the plasmids carrying spectinomycin (Spec) resistant gene. Three biological replicates were carried out for each condition and the standard errors were presented.

FIG. 8 Conditional essential genes in various pathways—A solid arrow represents a single enzymatic step while a dashed arrow represents multiple enzymatic steps. Abbreviation for CEG: pdxH: pyridoxine 5′-phosphate oxidase, aroA: 5-enolpyruvylshikimate-3-phosphate synthetase, aroB: 3-dehydroquinate synthase, aroC: chorismate synthase, pyrF, orotidine-5′-phosphate decarboxylase, proC: pyrroline-5-carboxylate reductase, argB: N-acetylglutamate kinase, argC: N-acetyl-gamma-glutamylphosphate reductase, argH: argininosuccinate lyase. Abbreviation for metabolites: TCA: tricarboxylic acid, G3P: Glyceraldehyde 3-phosphate, PEP: phosphoenolpyruvic acid, E4P: D-Erythrose 4-phosphate, α-KG: alpha-ketoglutaric acid, PPP: pentose phosphate pathway, PNP: pyridoxine-5′-phosphate, PLP: pyridoxal 5′-phosphate, UMP: Uridine monophosphate, UDP: Uridine diphosphate.

FIG. 9 Plasmid stability test for CAMPS with pyrF, proC and pdxH—The plasmid copy numbers of single-plasmid/host systems with or without IPTG induction (0.1 mM) were measured by qPCR. The T7-ADS-ispA-Cam-pyrF-p15AL2-1, T7-ADS-ispA-Cam-proC-p15AL2-4 and T7-ADS-ispA-Cam-pdxH-p15AL2-9 plasmids carrying conditional essential genes were introduced to engineered host with relevant gene knockout and cultured in minimum medium with glucose as carbon source.

FIG. 10 Introduction of multiple CEG carrying plasmids into engineered host—the illustration of the antibiotic-free method to introduce multiple CEG carrying plasmids into multiple CEG knockout strain with various modified minimum mediums.

FIGS. 11A-11B Plasmid stability of CAMPS—CAMPS were introduced into the mutant strain (MG1655, DE3, AaroABC) and cultured in minimum medium with glucose as carbon source. Various concentrations of IPTG (0.3 mM or 0.033 mM) were supplied to induce the genes carried by the plasmids to generate selection stress in certain conditions. (FIG. 11A) two CAMPS (MVA-213 and MVA-323) consist of three plasmids carrying the genes of MVA pathway and amorphadiene synthase: SAR: hmgS-hmgR-aroB, KKID: MVK-PMVK-MDV-idi, AA: ADS-ispA; (FIG. 11B) the plasmid copy numbers. Three biological replicates were carried out for each condition and the standard errors were presented.

FIGS. 12A-12C Amorphadiene production with CAMPS—(FIG. 12A) two CAMPS (MVA-213 and MVA-323) consist of three plasmids carrying the genes of MVA pathway and amorphadiene synthase: SAR: hmgS-hmgR-aroB, KKID: MVK-PMVK-MDV-idi, AA: ADS-ispA; Both systems were introduced into the native strain (MG1655, DE3) or the mutant strain (MG1655, DE3, ΔaroABC) for amorphadiene production. (FIG. 12B) various systems' amorphadiene yields when cultured in 2xPY++ medium supplied with three antibiotics to maintain the plasmids. (FIG. 12C) various systems' amorphadiene yields when cultured in minimum medium with either glucose or glycerol as carbon source without antibiotics. Four different IPTG concentrations were used to induce the expression of pathway genes.

FIG. 13 Stem-loop structure of RNA I in p15A like plasmids: Col El (SEQ ID NO: 4); p15A (SEQ ID NO: 5); RSF1030 (SEQ ID NO: 6); CloDF13 (SEQ ID NO: 7).

### DETAILED DESCRIPTION OF THE INVENTION

A description of example embodiments of the invention follows.

Described herein is the development of a multi-plasmid system (Compatible Antibiotic-free Multi-Plasmid System, CAMPS) for the expression of one or more (multiple; a plurality) nucleic acid sequences of interest (e.g., genes of interest (GOI)) carried by one or more (multiple; a plurality) multi-compatible plasmids in specially engineered host cells and grown in antibiotic-free medium. This was designed based on modifying a parental plasmid where the recognition sites controlling plasmid copy number were specifically engineered to produce a library of compatible plasmids. Thus, these plasmids share the same replication mechanism but vary in copy numbers. In order to maintain these multiple plasmids without using antibiotics, multiple conditional essential genes (CEG) from the host genome were grafted into these plasmids. As a result, all of these co-existing plasmids carrying the CEG were maintained in a host where the corresponding CEGs were knocked out from the host's genome during fermentation. The combination of multiple engineered compatible plasmids using the same replication mechanism co-existing in an antibiotic free fermentation condition has broad utility in metabolic engineering and synthetic biology.

Accordingly, in one aspect the invention is directed to a method of expressing a plurality (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.) of nucleic acid sequences comprising maintaining a (one or more) host cell comprising one or more, and preferably, two or more plasmids. In a particular aspect, the two or more plasmids are compatible. As used herein, compatible plasmids are plasmids that can stably co-exist in a host (e.g., host cell) and/or can grow under selection pressure. In a particular aspect, compatible plasmids can further grow without the use of antibiotics. The host cell lacks one or more conditional essential genes, and the two or more plasmids comprise the two or more nucleic acid sequences to be expressed and the sequences of the one or more conditional essential genes, and each plasmid further comprises an origin of replication (Ori) that is identical except for one or more loop sequences in the Ori of each plasmid. The host cell is maintained under conditions in which the two or more nucleic acid sequences and the one or more conditional essential genes are expressed in the host cell, thereby expressing the two or more nucleic acid sequences.

In another aspect, the invention is directed to a method of expressing two or more nucleic acid sequences comprising introducing into a host cell two or more plasmids, wherein (i) the host cell lacks one or more conditional essential genes and (ii) the two or more plasmids comprise the two or more nucleic acid sequences and the one or more conditional essential genes, and each plasmid comprises an origin of replication (Ori) that is identical except for one or more loop sequences in the Ori of each plasmid. The host cell is maintained under conditions in which the two or more nucleic acid sequences and the one or more conditional essential genes are expressed in the host cell, thereby expressing the two or more nucleic acid sequences.

In another aspect, the invention is directed to a host cell comprising two or more plasmids, wherein each plasmid comprises an origin of replication that is identical except for one or more loop sequences in the Ori of each plasmid. In some aspects, the host cell lacks one or more conditional essential genes. In other aspects, the plasmids can further comprise one or more conditional essential genes. In yet another aspect, the plasmids can comprise one or more conditional genes that are lacking in a (one or more) host cell.

In another aspect, the invention is directed to a host cell comprising two or more plasmids, wherein each plasmid comprises one or more CEGs. In another aspect, the invention is directed to a host cell wherein (i) the host cell lacks one or more conditional essential genes, and (ii) the two or more plasmids comprise the one or more conditional essential genes. The plasmids can further comprise an ORi that is identical except for one or more loop sequences in the Ori of each plasmid.

In yet another aspect, the invention is directed to a plurality of plasmids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 etc), such as a panel of plasmids. In one aspect, the plurality of plasmids comprises two or more plasmids.

In another aspect, each plasmid comprises an Ori that is identical except for one or more loop sequences in the Ori of each plasmid. The one or more plasmids can further comprise one or more CEGs. In a particular aspect, the plasmid comprises one or more CEGs that a (one or more) host cell lacks.

In another aspect, each plasmid comprises one or more conditional essential genes of a host cell. In particular aspects, the one or more plasmids comprise the one or more CEGs that a (one or more) host cell lacks. The plasmids can further comprise an Ori that is identical except for one or more loop sequences in the Ori of each plasmid.

In another aspect, the invention is directed to a system for expressing two or more nucleic acid sequences comprising a host cell and two or more plasmids, wherein the host cell lacks one or more conditional essential genes and the two or more plasmids comprise the one or more conditional essential genes, and each plasmid comprises an origin of replication (Ori) that is identical except for one or more loop sequences in the Ori of each plasmid.

In yet another embodiment, the invention is directed to a method of preparing a library of compatible plasmids comprising introducing into a host cell at least two plasmids wherein each plasmid comprises an origin of replication (Ori) that is identical except for one or more loop sequences in the On of each plasmid; and maintaining the host cell under conditions in which the plasmids are replicated in the host cell, thereby by preparing a library of compatible plasmids.

As described herein, the plasmids in the compositions and methods are compatible. As used herein, “compatible” plasmids refer to plasmids that when present in a host cell do not inhibit the replication of one another in the host cell. In addition or in the alternative, compatible plasmids are plasmids that can stably co-exist in a host (e.g., host cell) and/or can grow under selection pressure. In a particular aspect, compatible plasmids can further grow without the use of antibiotics. Each plasmid (e.g., in the host cell, the panel of plasmids, the system and/or the method) can further comprises an origin of replication (Ori), wherein the Ori of each plasmid is identical except for one or more nucleotides (bases) in the Ori sequence. In one aspect, the Ori of the two or more plasmids have a stem-loop structure wherein the two or more plasmids are identical except for one or more of the loop sequences in the Ori of each plasmid. The Ori of each plasmid can differ by one or more (e.g., 2, 3, 4, 5, 6, 7, 8, 9, 10, etc.) nucleotides. In a particular aspect, the Ori of each plasmid comprises 3 loops and one or more nucleotides in one or more of the loops (e.g., a first loop, a second loop and/or a third loop) of the On of each plasmid differs. In particular aspects, the Ori of each plasmid comprises 3 loops and the sequence of the second loop of the Ori of each plasmid differs (e.g., differs by one nucleotide; differs by two nucleotides, differs by three nucleotides, etc.). In one aspect, The method of any one of the preceding claims wherein at least one plasmid comprises a recognition site for RNase H (e.g., located near or next to the stem-loop structure of the Ori).

In addition, at least one plasmid in the host cell can further comprises one or more cloning sites, a nucleic acid sequence encoding a marker, and/or one or more nucleic acid sequences to be expressed in the host cell.

A variety of plasmids can be used in the compositions and methods provided herein. In particular aspect, the one or more plasmids comprise one or more of the following features; synthesize RNA primer for the initiation of replication (e.g., RNA II) with stem-loop structures (e.g., three); synthesize another antisense RNA for the regulation of replication (e.g., RNA I) with complementary sequences and stem-loop structures; synthesize initiator of replication (e.g., repZ protein) for the initiation of replication (e.g., RNA II) with stem-loop structures in its mRNA; synthesize another antisense RNA (e.g., Inc RNA) for the regulation of the translation of replication initiator (e.g., repZ protein) with complementary sequences and stem-loop structures; synthesize a polypeptide or RNA as the initiator for replication where the mRNA of the polypeptide or the RNA initiator has stem-loop structure(s); synthesize another antisense RNA with complementary sequences and stem-loop structure(s) to regulate the initiator; and/or comprise one or more loop sequences in an Ori that differ by one or more (2, 3, 4, 5, 6, 7, 8, 9, 10, etc.) nucleotides. Specific examples of plasmids include ColEl, pl5A, Incla (e.g., Collb-P9), pMV158, Inc18 (e.g., pIP501, pSM19035) and the like.

Any of a variety of host cells can be used in the methods and compositions provided herein. In one aspect, the host cell is an auxotroph host cell. For example, the host cell can be a prokaryotic host cell (e.g., E. coli).

As used herein, a “conditional essential gene” (CEG) is a gene of a host cell that is needed for growth of the host cell under one or more particular growth conditions (e.g., growth in minimal media), such as a metabolic gene. The one or more conditional essential genes (CEGs) are essential when the host cell is cultured in a selection culture media but not in a non-selection culture medium. As used herein a “selection (e.g., minimal) culture (growth) media” is a culture medium that lacks one or more essential components (e.g., the minimal necessities) for growth of the cell (e.g., a host cell that lacks one or more conditional essential genes). As used herein a “non-selection (e.g., rich) media correct is a media that includes one or more essential components for the growth of a cell (e.g., a host cell which lacks one or more conditional essential genes). In one aspect, the selection culture media comprises one or more sugars. In another aspect, the one or more sugars are glucose, glycerol or a combination thereof.

In other aspects, the one or more conditional essential genes encode one or more polypeptides in one or more biochemical pathways of the host cell such as a metabolic pathway of the host cell. In particular aspects, the one or more conditional essential genes encode one or more metabolic enzymes of the host cell. Examples of metabolic enzymes include aroA, aroB, aroC, pdxH, pyrF, proC, argB, arC, argH or a combination thereof.

Examples of CEGs are listed below.

List of Conditional Essential Genes that are Essential in Glucose or Glycerol Minimal Medium:

| | | | | | Purine and | | | |
|---|---|---|---|---|---|---|---|---|
| Amino acid | | | Cofactor | | pyrimidine | Regulatory | | |
| metabolism | | | production | | biosynthesis | proteins | Transport | Others |
| argA | hisB | pabA | bioA | thiC | carA | cysB | crr | atpA |
| argB | hisC | pabB | bioB | thiD | carB | furR | cysA | atpB |
| argC | hisD | pheA | bioC | thiE | guaA | hflD | cysU | atpC |
| argE | hisE | proA | bioD | thiG | guaB | leuL | fes | atpF |
| argG | hisG | proB | bioF | thiH | purA | metR | ptsI | atpG |
| argH | hisH | proC | bioH | coaA | purC | | | atpH |
| aroA | hisI | sera | cysG | coaB | purD | | | exoX |
| aroB | ilvA | serB | folB | coaC | purE | | | glmM |
| aroC | ilvB | serC | folP | coaE | purF | | | glnA |
| aroD | ilvC | thrA | iscC | | purH | | | glpD |
| aroE | ilvD | thrB | nadA | | purK | | | glpK |
| cysC | ilvE | thrC | nadB | | purI | | | gltA |
| cysD | leuA | trpA | nadC | | purM | | | lcd |
| cysE | leuB | trpB | panB | | pyrB | | | ppc |
| cysH | leuC | trpC | panC | | pyrC | | | prfB |
| cysI | leuD | trpD | panD | | pyrD | | | rpsU |
| cysJ | lysA | trpE | pdxA | | pyre | | | yhhK |
| cysK | metA | tyrA | pdxB | | pyrF | | | yjhS |
| cysN | metB | glnA | pdxH | | thyA | | | |
| cysP | metC | hisF | pdxJ | | | | | |
| cysQ | metE | | ubiE | | | | | |
| glyA | metF | | ubiG | | | | | |
| hisA | metL | | ubiH | | | | | |

1. Joyce, A. R., et al., Experimental and computational assessment of conditionally essential genes in Escherichia coli. J Bacteriol, 2006. 188(23): p. 8259-71.
2. Kim, J. and S. D. Copley, Why metabolic enzymes are essential or nonessential for growth of Escherichia coli K12 on glucose. Biochemistry, 2007. 46(44): p. 12501-11.

As described herein the host cell can lack one or more CEGs. In particular aspects, the host cell lacks 2, 3, 4, 5, 6, 7, 8, 9, 10, 11, 12, 13, 14, 15, 16, 17, 18, 19, 20, 21, 22, 23, 24, 25, 26, 27, 28, 29, 30 etc, CEGs. Each conditional essential gene can be inserted into a separate plasmid.

The methods provided herein can further comprising adding one or more moieties e.g., chemicals, produced by one or more polypeptides or intermediates thereof to the culture media, wherein the one or more polypeptides are encoded by the one or more conditional essential genes. In particular aspects, the moieties are needed for the growth of a host cell (e.g., an auxotroph host cell, for example, that lacks CEGs—the lack of CEGs results in the lack of essential metabolites for growth). In particular aspects, the one or moieties can be (i) the direct product (e.g., metabolite) of one or more polypeptides encoded by one or more CEGs that are or that can be converted to the essential metabolites lacking in the host cell (e.g., the auxotroph host cell) or (ii) the other metabolites that are or that can be converted to the essential metabolites (e.g., downstream of CEG and upstream of the essential metabolites).

In a particular aspect, the one or more intermediates are downstream in a biochemical (e.g., metabolic) pathway of the one or more CEGs. In other aspects, the one or more chemicals comprise one or more metabolites produced by the one or more polypeptides or intermediates thereof. For example, the one or more products can comprise pyridoxal 5′-phosphate (PLP), proline (PRO), uridine monophosphate (UMP), arginine (ARG), shikimate (SK), ornithine (OR) or a combination thereof.

In some aspects, the moiety (e.g., chemical such as a metabolite) for a CEG or a set of CEGs from a sequential metabolic pathway can be the direct product or the metabolite downstream of the CEG or the set of CEGs. Examples of such chemicals are provided below.

| CEG/CEGs | Metabolite | Description | |
|---|---|---|---|
| pdxH | pyridoxal 5′-phosphate | Single | Direct product |
| | (PLP) | CEG | |
| aroB | Shikimate (SK) | Single | Downstream |
| | | CEG | product |
| pyrF | uridine monophosphate | Single | Direct product |
| | (UMP) | CEG | |
| proC | proline (PRO) | Single | Direct product |
| | | CEG | |
| argB, argC | ornithine (OR) | Set of | Downstream |
| | | CEGs | product |
| argB, argC, argH | arginine (ARG) | Set of | Direct product |
| | | CEGs | |
| ubiG | Ubiquinone | Single | Direct product |
| | | CEG | |
| nadA, nadB, nadC | Nicotinamide adenine | Set of | Downstream |
| | dinucleotide (NAD) | CEGs | product |
| bioA, bioB, bioC, | biotin | Set of | Direct product |
| bioD, bioF, bioH | | CEGs | |
| thiC, thiD, thiE, | Thiamine | Set of | Downstream |
| thiG, thiH | | CEGs | product |
| folB, folP | Tetrahydrofolic acid | Set of | Downstream |
| | | CEGs | product |
| purC, purD, purE, | inosine monophosphate | Set of | Direct product |
| purF, purI, purM, | (IMP) | CEGs | |
| purK, purH | | | |
| guaB, guaA | guanosine | Set of | Direct product |
| | Monophosphate (GMP) | CEGs | |
| aroA, aroB, aroC, | Chorismic acid | Set of | Direct product |
| aroD, aroE | | CEGs | |
| cysC, cysD, cysE, | cysteine | Set of | Direct product |
| cysH, cyst, cysN | | CEGs | |
| glyA | glycine | Single | Direct product |
| | | CEG | |
| hisA, hisB, hisC, | histidine | Set of | Direct product |
| hisD, hisE, hisF, | | CEGs | |
| hisG, hisH, hisI | | | |
| leuA, leuB, leuC, | leucine | Set of | Downstream |
| leuD | | CEGs | product |
| lysA | lysine | Single | Direct product |
| | | CEG | |
| trpA, trpB, trpC, | tryptophan | Set of | Direct product |
| trpD, trpE | | CEGs | |
| pheA | phenylalanine | Single | Downstream |
| | | CEG | product |
| tyrA | tyrosine | Single | Downstream |
| | | CEG | product |
| sera, serB, serC | serine | Set of | Direct product |
| | | CEGs | |
| ilvA, ilvC, ilvD, ilvE | valine | Set of | Direct product |
| | | CEGs | |

In particular aspects of the methods provided herein, the two or more nucleic acid are expressed under antibiotic-free conditions. In other aspects, the one or more nucleic acid sequence is one or more genes. In yet other aspects, the one or more genes are overexpressed by the host cell.

### EXEMPLIFICATION

A scalable compatible antibiotic-free multi-plasmid system (CAMPS) for metabolic engineering and synthetic biology

Results

Overview of CAMPS

To develop this scalable system (CAMPS) for metabolic engineering and synthetic biology, an integrated: 1) compatible plasmids and 2) conditional host cell system was designed and engineered specifically. These two components functions as one to achieve the desired outcome of an antibiotic free multi-plasmid expression system for the scale-up production of valued compounds by controlling the expressions of multiple genes (FIG. I).

Engineering of Compatible Plasmids

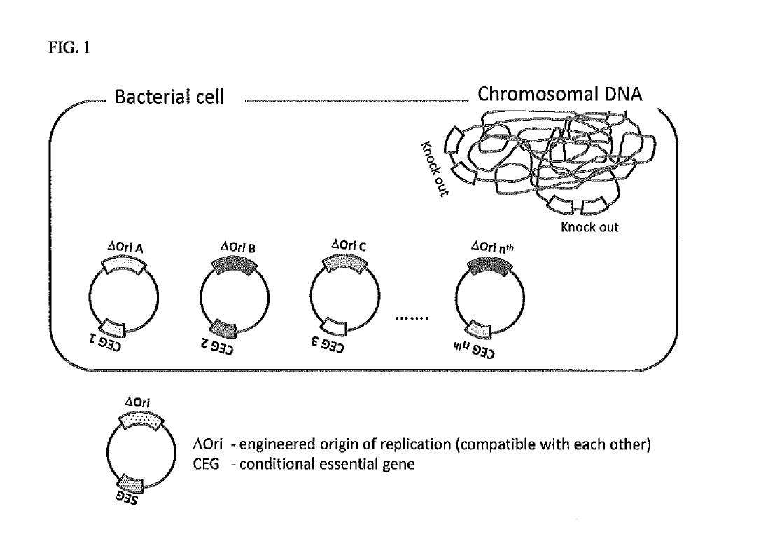

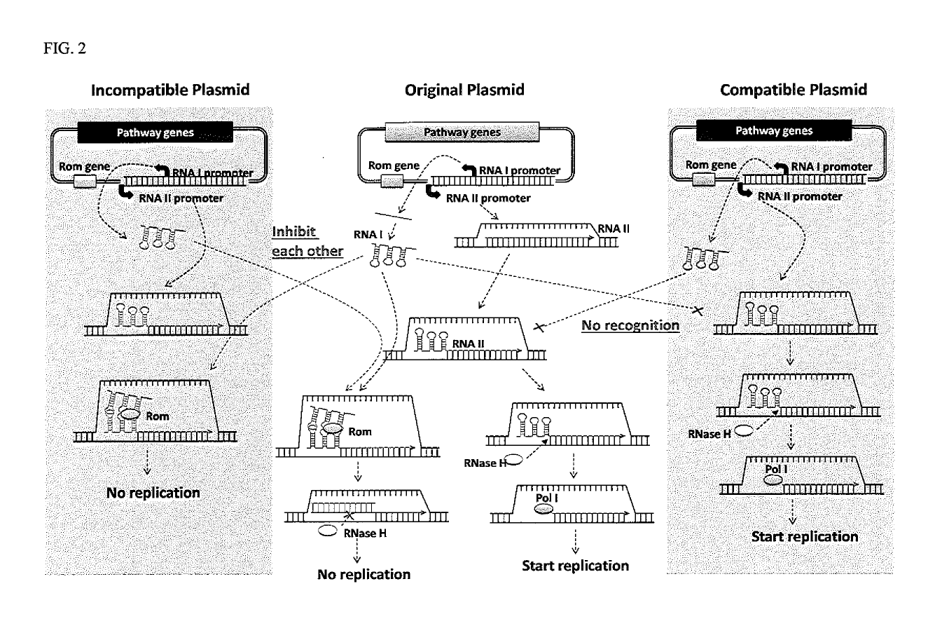

The replication and copy number of a class of plasmid (such as P15A and ColE1) is tightly controlled by the trans-acting factor RNA I as illustrated in FIG. 1 (Original plasmid). The replication of a plasmid starts with the synthesis of the RNA primer—RNA II through the recognition of its promoter by RNA polymerase. The synthesized RNA II (in blue) will hybridize with the template DNA (the plasmid, in black) except for sequences around its 3′ prime which will form stable stem-loop secondary structures. The RNA II will then be cleaved by RNase H (green ellipse) to generate a hydroxyl group recognizable by DNA polymerase I (Pol I, purple ellipse) which can synthesize the DNA primer to initiate replication. On the other hand, a promoter on the opposite strand will produce RNA I (in blue)—a short RNA complementary to the 3′ prime sequences of RNA II. The RNA I and RNA II will form symmetrical stem-loop structures exposing complete complementary sequences at each loop. Those sequences promote the annealing of RNA I and RNA II with the aid of Rom protein (red ellipse) to form a RNA-RNA hybrid which blocks the cleavage site for RNase H and in turn stops the replication. Inside each cell, the concentration of the inhibitor—RNA I, will increase with the increasing number of plasmid which forms a dynamic feedback process controlling the plasmid copy number. Thus, by modulating the promoters and expression levels of these RNAs, it is possible to change the copy numbers of the plasmids. Furthermore, if another plasmid with the same On site coexists, both plasmids will generate identical RNA I and RNA II sequences and these will compete for interactions and inhibit the replication of each other (FIG. 2, Incompatible plasmid). This phenomenon called ‘incompatibility’ will destabilize the copy numbers of both plasmids resulting in the loss of a less favorable one (due to size, sequences, GOI etc).

Described herein is the generation of a set of plasmids (FIG. 2, Compatible plasmid) that avoids the incompatibility issue. A panel of plasmids that do not exist in nature with sites of modifications that were unique were generated. As the desired sites to be modified could not be predicted in silica with precision, all the designs were empirically validated.

The step in replication, which can result in plasmid incompatibility, is the annealing of RNA I and RNA II to the recognition sites. As shown herein, altering the loop sequences made it possible to modify the recognition (FIG. 2,Compatible plasmid, red sequences) step. These engineered plasmids would then be compatible with the original parental plasmid.

Precisely which nucleotide and the number of nucleotides on the loop to be modified was determined empirically as the effect of loop sequence on complex stability did not appear to vary simply with the number of potential Watson-Crick base-pairs that can be formed. For example, sequences that produce inverted loops can result in extremely low copy numbers. In addition, single nucleotide changes in the sequences of RNA I/RNA II overlap can alter the affinity of their interaction but can maintain the complementarity of the sequences. This built-in tolerance to point mutations is thought to facilitate the mutational fine-tuning of the system to maintain the existence of compatible plasmids and exclude incompatible plasmids with only minor changes. As a result, with single nucleotide changes, the incompatibility properties can only be altered to a limited extent. Thus, to modify the sequences to achieve compatibility of multiple plasmids experimentation was needed since it was not obvious by in silico prediction.

To create a large plasmid library whereby members are not only compatible with the parental plasmid but also with other members in the library, the engineered sites on the loops should be sufficiently different between different plasmids. Targeting at the loop sequences, a number of modifications to enable these plasmids to be compatible were empirically identified,

At the same time, because the synthesis of RNA I and RNA II of the same plasmid share the same DNA template, the copy number control mechanism can be retained for each member. Thus, by engineering the Ori sites, plasmids that shared the same replication mechanism as the parental which were similarly regulated upon environmental changes were generated.

To demonstrate the idea, the second loop in the stem-loop structures of the origin of replication of the vector pAC vector, p15A Ori was generated. A series of new plasmids with unique 2nd loop sequences were constructed by site-directed mutagenesis and the screened plasmids showed a range of copy numbers. Selected plasmids were then tested and confirmed to be compatibility with the original one and with each other.

Construction of Artificial Multi-Compatible Plasmids

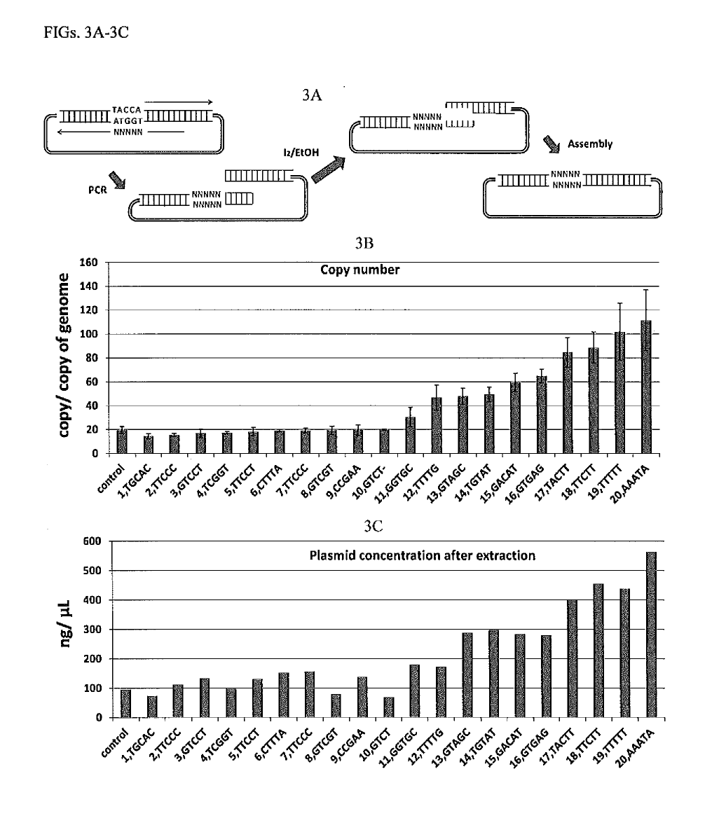

In order to construct multiple compatible plasmids based on the designs described above, site-directed mutagenesis was carried out to mutate the 2nd loop of p15A Ori (FIG. 3A). A pair of primers with 5 degenerate bases targeting the loop sequences (TGGTA) was used to amplify T7-ADS-ispA-Cam-p15A-PAC plasmid. Both primers were pre-modified with thiophosphate and the PCR products were specifically cleaved with iodine/ethanol solution to generate single strand DNA overhangs. The mutated plasmids were formed by the annealing of two complementary overhangs and then transformed into E. coli. After overnight culture, twenty randomly selected colonies were amplified and the plasmids were sequenced. Only two plasmids had the same sequences at the 2nd loop: TTCCC (FIG. 3B, number 2 and 8) and one plasmid had 4 bps instead of 5 (FIG. 3B, number 10) possibly due to the impurity of the degenerate primer. Those new Ori sites were named p15AL2-1 to p15AL2-20. The plasmids were then separately transformed into MG1655 DE3 strain for copy number measurement. For each plasmid, two random colonies were picked and the copy numbers were measured by qPCR (FIG. 3B) by normalizing to genomic DNA. The concentrations of the extracted plasmids were similar to the copy numbers measured by qPCR (FIG. 3C). Half of the panel of plasmids had similar copy numbers (16-20 copies per copy of genome) when compared to the original parental plasmid (FIG. 3C, control) while the other half had higher copy numbers from 20 to 110 copies per copy of genome. Thus, a library of newly engineered plasmids with varying copies was constructed.

Compatibility Test of Multi-Compatible Plasmids

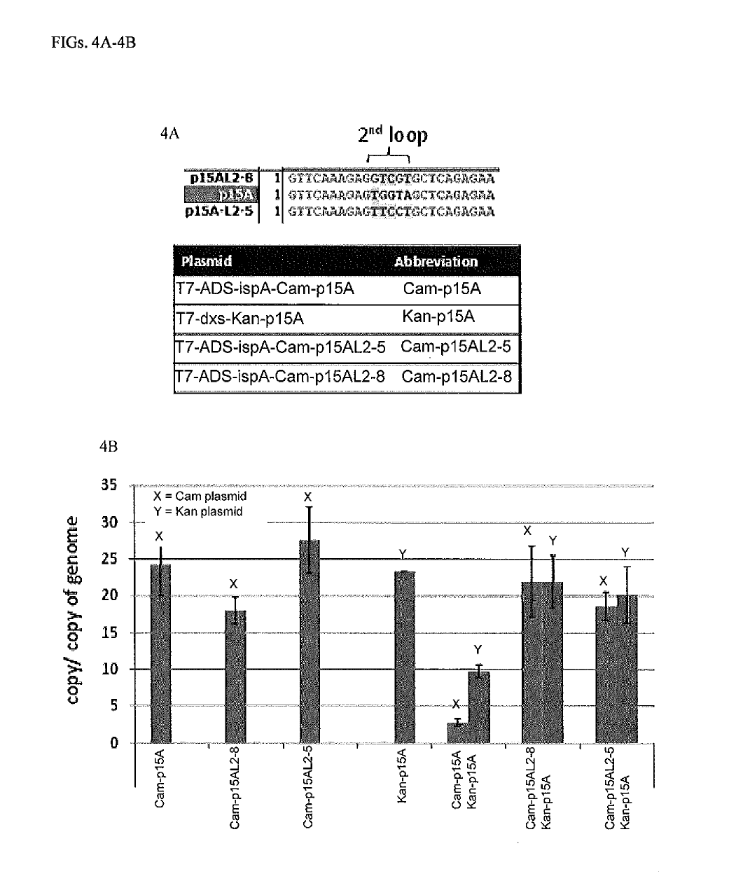

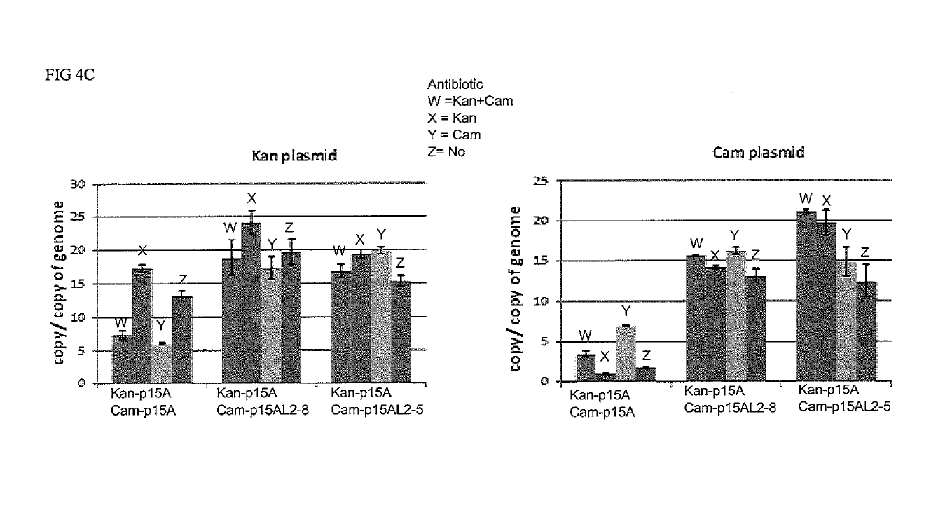

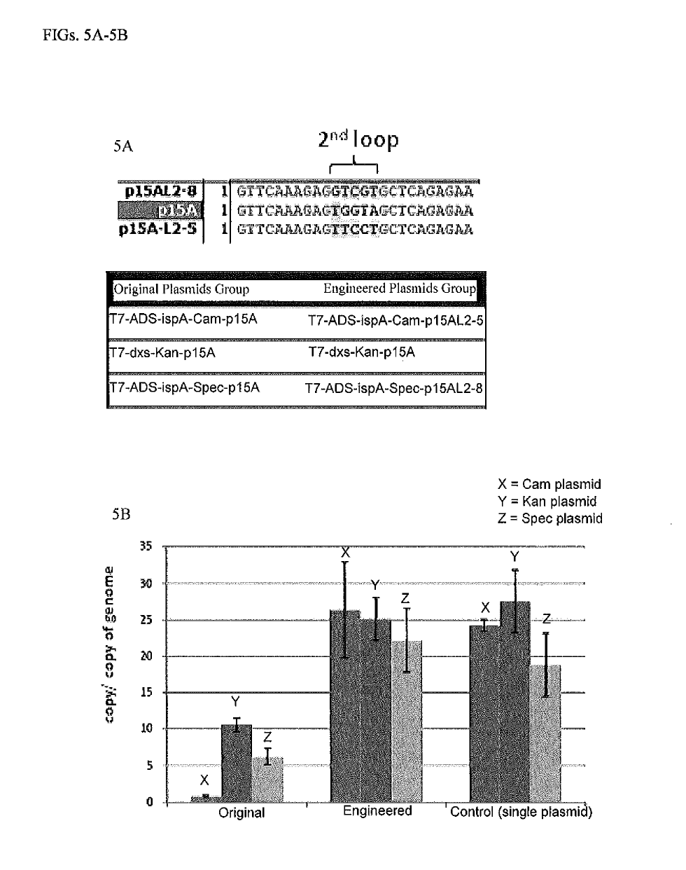

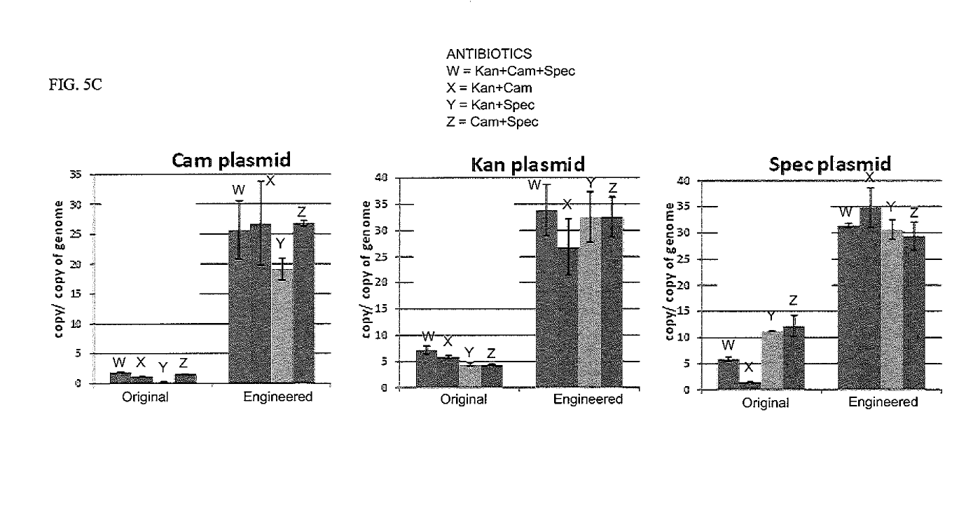

As a proof-of-concept, multiple plasmids with different engineered On sites were tested for compatibility. The original site (p15A) and two engineered sites (p15AL2-5 and p15AL2-8) were inserted into different plasmids together with various antibiotic resistant genes (FIG. 4A; FIG. 5A). Plasmids with these engineered sites have similar copy numbers in cells. These plasmids carried antibiotic resistant genes simply to enable the ease of selecting for the multiple plasmids.

Firstly, a kanamycin (Kan) resistant plasmid with the original Ori site (T7-dxs-Kan-p15A) was co-transformed with each of the three Ori sites carrying the chloramphenicol (Cam) resistant plasmid (T7-ADS-ispA-Cam-p15A, T7-ADS-ispA-Cam-p15AL2-5 or T7-ADS-ispA-Cam-p15AL2-8). Both Kan and Cam were used initially to maintain the plasmids. In subsequent constructions, these would be substituted with CEG and conditionally rescued with the appropriate products (see below section). A comparison of the copy numbers in cells carrying single plasmid or dual-plasmid were carried out (FIG. 4B). As expected, cells with dual-plasmid of the same Ori sequences (Cam-p15A or Kan-p15A) showed significantly lower copy numbers as compared to cells carrying just a single plasmid (Kan-p15A) even when antibiotics were supplied to force the maintenance of both plasmids. The behaviors of these dual-plasmid systems were then further studied in conditions where Kan, Cam or both antibiotics were removed from the medium to release the selection pressure (FIG. 4C). The dual-plasmid cells with different Ori sites (engineered as described above) had identical performances in all conditions proving the plasmid incompatibility problem and that there was no interaction between the engineered Ori with the original one. Instead, the plasmids with identical On (Kan-p15A and Camp-p15A) showed variable copy numbers. For example, the removal of Cam (Kan or No antibiotics conditions) induced the loss of Cam-p15A plasmid (FIG. 4C, Cam plasmid, Kan-p15A/Cam-p15A group) and at the same time resulted in a higher copy number for the competing plasmid (Kan-p15A) comparing to the dual-antibiotic condition (FIG. 4C, Kan plasmid, Kan—p15A/Cam—p15A group). These evidences support the idea that plasmids with engineered novel On sites were compatible with the original parental plasmid, co-existing in the same cells.

To further validate the compatibility between two artificial Ori sites where the loop sequences differed only by 2 bases, cells carrying two groups of plasmids differ only by the On sites were compared. Both groups had three plasmids with either kanamycin (Kan), chloramphenicol (Cam) or spectinomycin (Spec) resistant genes respectively. All the plasmids in the “Original group” (T7-ADS-ispA-Cam-p15A, T7-dxs-Kan-p15A and T7-ADS-ispA-Spec-p15A) had p15A as their Ori sites while the “Engineered group” (T7-ADS-ispA-Cam-p15AL2-5, T7-dxs-Kan-p15A and T7-ADS-ispA-Spec-p15AL2-8) were plasmids with different On sites (FIG. 5A). As expected, in the presence of all three antibiotics, the copy numbers of the plasmid in the “Original group” were significantly lower than with cells harboring a single plasmid (control), consistent with the idea that the replication of these 3 plasmids (with identical Ori sequence) were affected by the presence of each other (FIG. 5B) while the “Engineered Group” was unaffected. Furthermore, in the absence of an antibiotic, the copy numbers of the three plasmids varied in the “Original group” as compared to the “Engineered group” where the copy numbers were consistently high (FIG. 5C). The results of this triple-plasmid study confirmed that plasmids with engineered Ori sites were compatible and can co-exist in the cells. Based on this, a multi-compatible plasmid system containing large number of plasmids can be easily established by introducing other mutations to the stem-loop sequences to create artificial Ori sites, which will be invaluable for studies involving the manipulation of multiple genes or pathways.

Design of Conditional Host Cell System

As discussed above, a significant disadvantage of using plasmid is the potential loss during cell growth and this can be averted by selection pressure using antibiotics. Various approaches to maintaining single plasmids without the use of antibiotics in bacteria include the manipulation of essential genes such as dnpD, glyA, fabI, murA, acpP or the use of antidote/poison system. With multiple plasmids, however, the situation is more complex and will depend on the products to be manufactured. As yet, there has not been any demonstration of the use of these methods for the maintenance of multi-plasmid where multiple GOI are required to be simultaneously expressed and the plasmids stably maintained simultaneously.

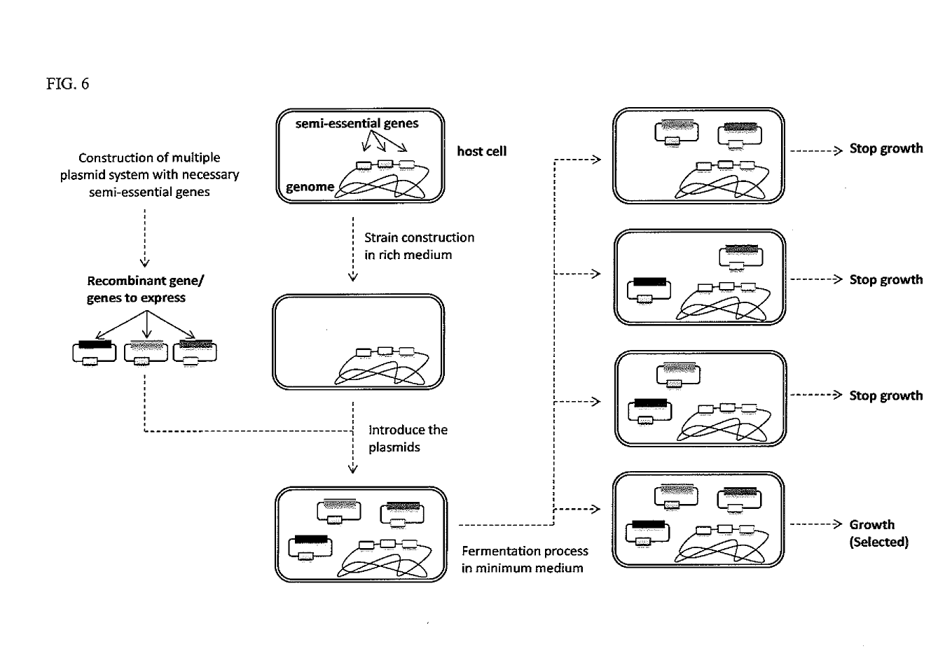

A universal workflow to construct a multiple-plasmid/host system by generating auxotroph host and complementing with conditional essential genes (CEG) was developed. By knocking out multiple CEG in a host genome and at the same time placing them separately on various plasmids, only cells with all the essential components—the genome and all the plasmids with CEG will survive (FIG. 6). A significant challenge was the construction of these multiple knockout strains as removing CEG will usually be non-viable in the minimal medium used. With cost consideration, industrial fermentations usually are carried out in minimal medium with either glucose or glycerol as carbon source. Using genes that were essential in minimal medium but were dispensable in rich medium allowed a way to conveniently generate multiple knockout strains by culturing the cells in rich medium (FIG. 6).

Not all metabolic enzymes are essential for growth. Hence, the knock-out of an enzyme in a pathway can be compensated by alternative biosynthetic pathways or the availability of isoenzymes, the existence of alternative enzymes and even broad specificity or multifunctional enzymes. Whether certain isozymes and alternative enzymes can complement for the missing one depends also on the expression levels and active states of the enzymes in that particular condition of growth.

The selection of CEG was not easily predicted from current knowledge and available resources. These databases contained collections of single but not multiple genes acting as conditionally or totally essential for growth in certain conditions. However, with the CAMPS knock-out of multiple CEG allowed the insertions and rescue of growth by using multiple plasmids with complementing CEG. Multiple CEG (e.g., metabolic enzymes) were knocked-out for performance of the strain in both growth and productivity.

With multiple co-existing compatible plasmids, auxotrophy could be achieved by using multiple CEG in series or in parallel of metabolic pathways. The criteria for the selection of either of these two approaches may be guided by a priori knowledge but was empirically established to provide a flexible, convenient and robust plasmid based platform for metabolic engineering and synthetic biology.

In order to conveniently introduce large numbers of CAMPS plasmids, sets of one to three CEG were distributed into subgroups based on their functionalities. For each subgroup, instead of using the complex rich medium, the auxotroph was rescued by the supplementation of specific metabolite that is economic and accessible to the cell. As a result, multiple CAMPS plasmids were sequentially or separately introduced into corresponding complementary host as subgroups, increasing the flexibility of pathway engineering.

To demonstrate the use of an enzyme in a serial biochemical pathway, three CEG from a pool of 94 genes that were thought to be essential in both glycerol and glucose minimum mediums but not in rich medium were selected and separately placed into three plasmids. The plasmids with these CEG (ΔaroA, ΔaroB and ΔaroC) were then shown to be stably maintained in single-plasmid/host, dual-plasmid/host and triple-plasmid/host systems.

Construction of Antibiotic-Free Multiple-Plasmid/Host System

To demonstrate the proposed work flow for the establishment of multiple-plasmid/host system (FIG. 6), three CEG—aroA (5-enolpyruvylshikimate-3-phosphate synthetase), aroB (3-Dehydroquinate synthase) and aroC (Chorismate synthase)—in a sequential aromatic amino acid biosynthesis pathway, were selected and inserted into three independent plasmids with different Ori sites: T7-ADS-Cam-aroB-p15A, T7-dxs-Kan-aroA-pET and T7-CYP450-CPR-Spec-aroC-pCL plasmid. Host strains with single or multiple CEG knockouts (Table 1) were constructed in 2xPY medium (rich medium).

TABLE 1
Strains used in the study

| Name | Genotype |
|---|---|
| MG1655 (DE3) | F⁻ λ⁻ ilvG- rfb-50 rph-1 |
| MG1655 (DE3, ΔaroA) | F⁻ λ⁻ ilvG- rfb-50 rph-1 ΔaroA |
| MG1655 (DE3, ΔaroB) | F⁻ λ⁻ ilvG- rfb-50 rph-1 ΔaroB |
| MG1655 (DE3, ΔaroC) | F⁻ λ⁻ ilvG- rfb-50 rph-1 ΔaroC |
| MG1655 (DE3, ΔaroBC) | F⁻ λ⁻ ilvG- rfb-50 rph-1 ΔaroB ΔaroC |
| MG1655 (DE3, ΔaroABC) | F⁻ λ⁻ ilvG- rfb-50 rph-1 ΔaroA ΔaroB ΔaroC |
| MG1655 (DE3, ΔpdxH) | F⁻ λ⁻ ilvG- rfb-50 rph-1 ΔpdxH |
| MG1655 (DE3, ΔproC) | F⁻ λ⁻ ilvG- rfb-50 rph-1 ΔproC |
| MG1655 (DE3, ΔpyrF) | F⁻ λ⁻ ilvG- rfb-50 rph-1 ΔpyrF |
| MG1655 (DE3, ΔargBCH) | F⁻ λ⁻ ilvG- rfb-50 rph-1 ΔargBCH |
| MG1655 (DE3, ΔaroABC, | F⁻ λ⁻ ilvG- rfb-50 rph-1 ΔaroA ΔaroB ΔaroC |
| ΔpdxH) | ΔpdxH |
| MG1655 (DE3, ΔaroABC, | F⁻ λ⁻ ilvG- rfb-50 rph-1 ΔaroA ΔaroB ΔaroC |
| ΔproC) | ΔproC |
| MG1655 (DE3, ΔaroABC, | F⁻ λ⁻ ilvG- rfb-50 rph-1 ΔaroA ΔaroB ΔaroC |
| ΔpyrF) | ΔpyrF |
| MG1655 (DE3, ΔaroABC, | F⁻ λ⁻ ilvG- rfb-50 rph-1 ΔaroA ΔaroB ΔaroC |
| ΔargBCH) | ΔargBCH |

The host and plasmid combinations were then processed for survival test. The strains harboring various plasmids were initially generated in rich medium with the addition of antibiotics. If there was no observation of growth after 90 hour incubation at 37° C. with shaking in the specified medium without antibiotics, the strain was considered to be non-viable. Based on the survival test, all the single knockout strains (MG1655 (DE3, ΔaroA); MG1655 (DE3, ΔaroB) and MG1655 (DE3, ΔaroC)) did not grow in minimum medium with either glycerol or glucose as carbon source confirming their essentialness in minimum medium (Table 2). On the other hand, the growth was only rescued by supplying the relevant plasmid such as inserting T7-ADS-Cam-aroB-p15A plasmid expressing aroB into MG1655 (DE3, ΔaroB) strain. At the same time, there was no growth of MG1655 (DE3, ΔaroB) strain with T7-ADS-Cam-p15A plasmid in minimum medium confirming that the rescue effects were due to the expressions of the CEG in the plasmid. No rescue activity was observed by the overexpression of irrelevant CEG by other plasmids confirming that the selected CEG could not serve as isoenzymes for each other. Similar observations were also observed in other multiple-plasmid systems (Table 3). In minimum medium, the MG1655, (DE3, ΔaroB) strain could only replicate when carrying both T7-ADS-Cam-aroB-p15A and T7-CYP450-CPR-Spec-aroC-pCL plasmids while the MG1655 (DE3, ΔaroABC) strain needed to carry all three plasmids (T7-dxs-Kan-aroA-pET, T7-ADS-Cam-aroB-p15A and T7-CYP450-CPR-Spec-aroC-pCL) for survival. Thus, these engineered hosts could only survive if they carried the relevant plasmids in minimum medium.

TABLE 2
Survival test of single-plasmid/ host system

| | | Strain | | | |
|---|---|---|---|---|---|
| Condition | | | MG1655, | MG1655, | MG1655, |
| Growth | | MG1655, | DE3, | DE3, | DE3, |
| medium | Plasmid | DE3 | ΔaroA | ΔaroB | ΔaroC |
| 2xPY medium | — | Yes | Yes | Yes | Yes |
| Minimum | — | Yes | No | No | No |
| medium (with | T7-ADS-Cam-p 15A | Yes | No | No | No |
| glucose or | T7-ADS-Cam-aroB-pl 5A | Yes | No | Yes | No |
| glycerol as | T7-dxs-Kan-pET | Yes | No | No | No |
| carbon source) | T7-dxs-Kan-aroA-pET | Yes | Yes | No | No |
| | T7-CYP450-CPR-Spec-pCL | Yes | No | No | No |
| | T7-CYP450-CPR-Spec-aroC-pCL | Yes | No | No | Yes |

Yes: Growth,
No: No growth

TABLE 3
Survival test of multi-plasmid/host system

| | | Strain | |
|---|---|---|---|
| Condition | | MG1655, | MG1655, |
| Growth | | DE3, | DE3, |
| medium | Plasmid | ΔaroBC | ΔaroABC |
| Minimum | — | No | No |
| medium (with | T7-dxs-Kan-aroA-pET | No | No |
| glucose or | T7-ADS-Cam-aroB-PISA | | |
| glycerol as | T7-ADS-Cam-aroB-p15A | Yes | No |
| carbon source) | T7-CYP450-CPR-Spec-aroC-pCL | | |
| | T7-dxs-Kan-aroA-pET | No | No |
| | T7-CYP450-CPR-Spec-aroC-pCL | | |
| | T7-dxs-Kan-aroA-pET | Yes | Yes |
| | T7-ADS-Cam-aroB-p15A | | |
| | T7-CYP450-CPR-Spec-aroC-pCL | | |

Yes: Growth,
No: No growth

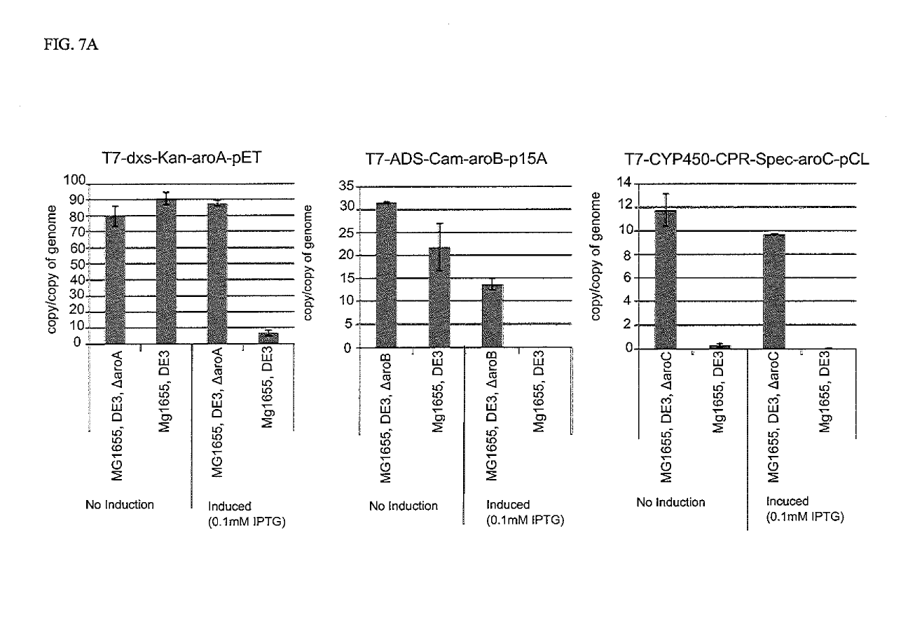

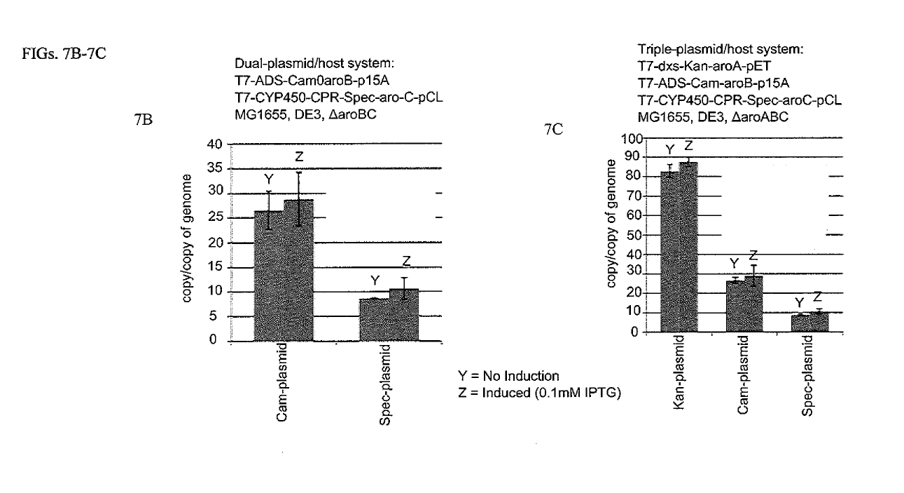

Extending the study, the plasmid copy numbers were measured after 2 day growth in minimum medium with no antibiotic selection. IPTG—the inducer for T7 promoter was supplied in some of the conditions to create selection pressure to cells harboring recombinant proteins expressed under the control of T7 promoter. For single-plasmid/host system, the T7-dxs-Kan-aroA-pET plasmid expressing E. coli native enzyme (dxs—1-deoxyxylulose-5-phosphate synthase) and T7-ADS-Cam-aroB-p15A plasmid expressing a plant gene (ADS—amorphadiene synthase) could be retained in both native strain (MG1655 (DE3)) and the modified strain (MG1655 (DE3, ΔaroA) or MG1655 (DE3, ΔaroB)), where induction of gene expression by IPTG were carried out. However, after induction (0.1mM IPTG) both plasmids were lost in the native strain but well retained in the modified strains (FIG. 7A). The T7-CYP450-CPR-Spec-aroC-pCL plasmid expressing cytochrome p450 enzymes from plant, a class of membrane binding enzymes known to cause stress to bacteria, were lost in the native strain even without IPTG induction while the modified strain retained the plasmid under uninduced or induced conditions (FIG. 7A). The dual-plasmid/host (FIG. 7B) and triple-plasmid/host (FIG. 7C) systems were also able to retain their plasmids in minimum medium without antibiotic. These results demonstrated the stable maintenance of multiple-plasmids in engineered host without the need of antibiotic.

Demonstration of the Use of CEG from Other Pathways

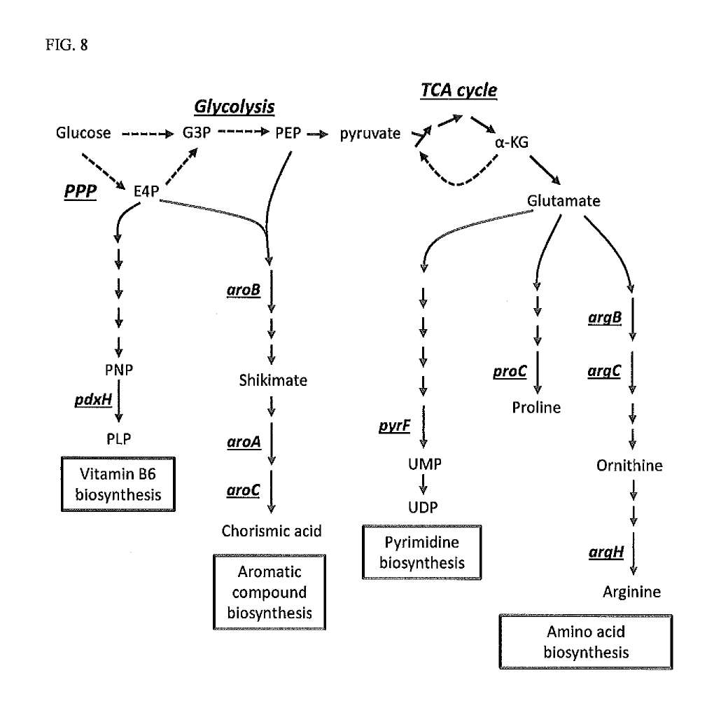

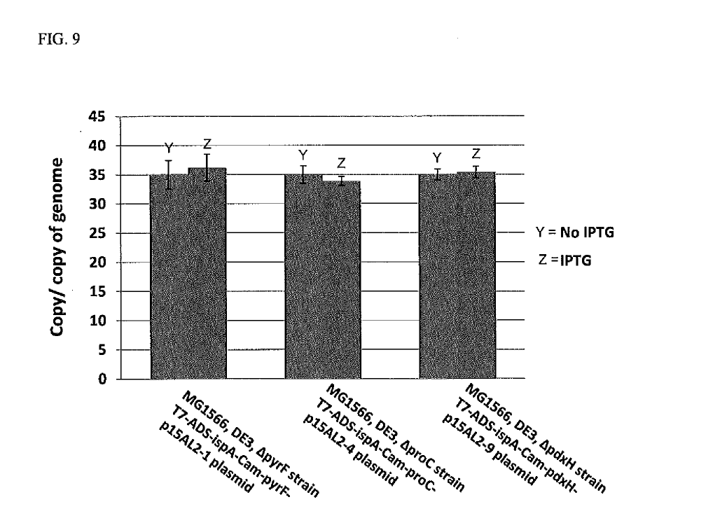

Three CEG (aroA, aroB and aroC, FIG. 8) were all selected from the aromatic compound synthesis pathway (FIG. 8). In order to further demonstrate the effectiveness of antibiotic-free plasmid/host system using CEG, genes from other pathways that synthesize conditional essential compounds including co-factors, amino acids and nuclear acids that could be rescued by growth in rich medium were grafted from the E. coli genome to various CAMPS plasmids (FIG. 8). The pdxH gene encoding the last step of PLP (pyridoxal 5′-phosphate) biosynthesis, the pyrF gene encoding the UMP synthase andproC gene encoding the last step of proline biosynthesis were placed into the following engineered vectors—p15AL2-9, p15AL2-4 and p15AL2-4, respectively (Table 4). The viability test confirmed that the engineered host with CEG knockout could only grow in the minimum medium (with glucose as carbon source) in the presence of the appropriate CAMPS plasmid (Table 4, strain 1-3). The plasmids stability were also validated by comparing the plasmid copy number of these three strains cultured in minimum medium with or without selection stress (induction of recombinant genes ADS and ispA expression with 0.1 mM IPTG) where there was no difference observed in these two conditions (FIG. 9).

Moreover, the multiple-knockout strains without aroA, aroB, araC genes and pdxH, pyrF, or proC gene in the genome could only survive when all four plasmids carrying necessary CEG were present (Table 4, strain 5-6). And the strain where the argBCH operon encoding three CEG in the biosynthetic pathway of arginine was knocked out can only grow in the presence of all the three CAMPS plasmids—T7-ADS-ispA-Cam-argB-p15AL2-10, T7-ADS-ispA-Cam-argC-p15AL2-6 and T7-ADS-ispA-Cam-argH-p15AL2-11 (Table 4, strain 4). These results lend further evidence that the multiple antibiotic-free plasmids expressing the CEG complemented the host where these genes were deleted.

TABLE 4
Survival test of plasmid(s)1 host systems using novel CEG

| Plasmid | | |
|---|---|---|
| | Strain 1 | |
| | MG1655, | |
| | DE3 | MG1655, DE3, ΔpdxH |
| — | Yes | No |
| T7-ADS-ispA-Cam-pdxF1-p15AL2-9 | Yes | No |
| | Strain 2 | |
| | MG1655, | |
| | DE3 | MG1655, DE3, ΔproC |
| — | Yes | No |
| T7-ADS-ispA-Cam-proC-p15AL2-4 | Yes | No |
| | Strain 3 | |
| | MG1655, | |
| | DE3 | MG1655, DE3, ΔpyrF |
| — | Yes | No |
| T7-ADS-ispA-Cam-pyrF-p15AL2-1 | Yes | No |
| | Strain 4 | |
| | MG1655, | MG1655, DE3, |
| | DE3 | ΔargBCH |
| — | Yes | No |
| T7-ADS-ispA-Cam-argB-pl5AL2-10 | Yes | No |
| T7-ADS-ispA-Cam-argC-p15AL2-6 | | |
| T7-ADS-ispA-Cam-argH-p15AL2-11 | | |
| | Strain 5 | |
| | MG1655, | MG1655, DE3, |
| | DE3 | ΔaroABC, ΔpdxH |
| — | Yes | No |
| T7-ADS-ispA-Cam-pdxH-p15AL2-9 | Yes | No |
| T7-dxs-Kan-aroA-pET | | |
| T7-ADS-Cam-aroB-p15A | | |
| T7-CYP450-CPR-Spec-aroC-pCL | | |
| | Strain 6 | |
| | MG1655, | MG1655, DE3, |
| | DE3 | ΔaroABC, ΔproC |
| — | Yes | No |
| T7-ADS-ispA-Cam-proC-p15AL2-4 | Yes | No |
| T7-dxs-Kan-aroA-pET | | |
| T7-ADS-Cam-aroB-p15A | | |
| T7-CYP450-CPR-Spec-araC-pCL | | |
| | Strain 7 | |
| | MG1655, | MG1655, DE3, |
| | DE3 | ΔaroABC, ΔpyrF |
| — | Yes | No |
| T7-ADS-ispA-Cam-pyrF-p15AL2-1 | Yes | No |
| T7-dxs-Kan-aroA-pET | | |
| T7-ADS-Cam-aroB-p15A | | |
| T7-CYP450-CPR-Spec-aroC-pCL | | |

Yes: Growth,
No: No growth.
The growth test was carried out in minimum medium with glucose as carbon source.

Introduction of Multiple CEG Carrying Plasmids into Engineered Host

The construction of multiple CEG knockout host for antibiotic-free system can be achieved with the procedures described in FIG. 6. However, the assembly of CAMPS faced significant challenge where multiple CEG carrying plasmids were required to be transformed into the same host. The co-transformation of plasmids in to E. coli is technically difficult, especially for large plasmids encoding multiple pathway genes. Practically, at most two plasmids can be introduced into the host at once and this will not meet the requirement for CAMPS. One way to transform multiple plasmids into the host is to do it one plasmid at a time using different antibiotics for selection at each insertion. Unfortunately, there is only limited number of antibiotics available and having multiple antibiotic resistant genes will incur metabolic burden and hence, is not practical.

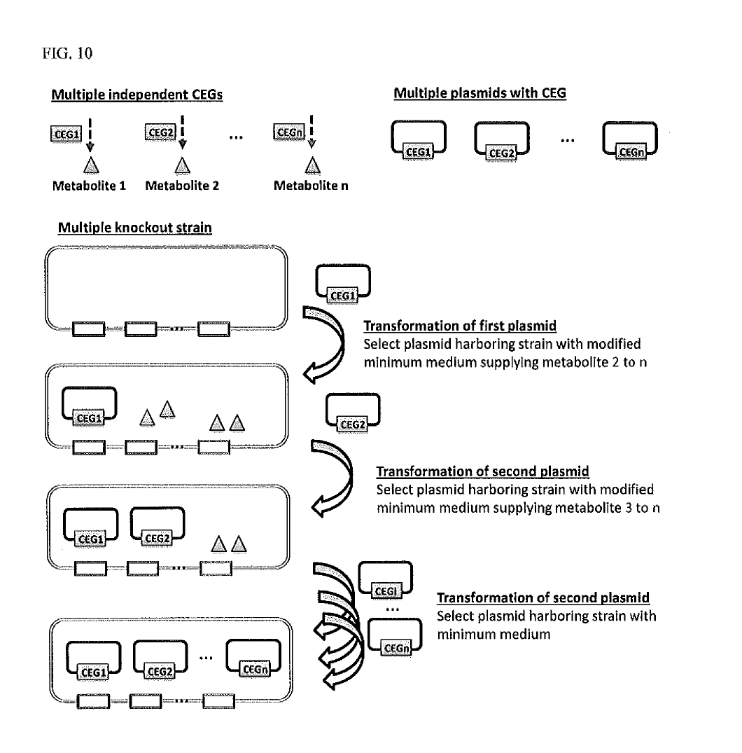

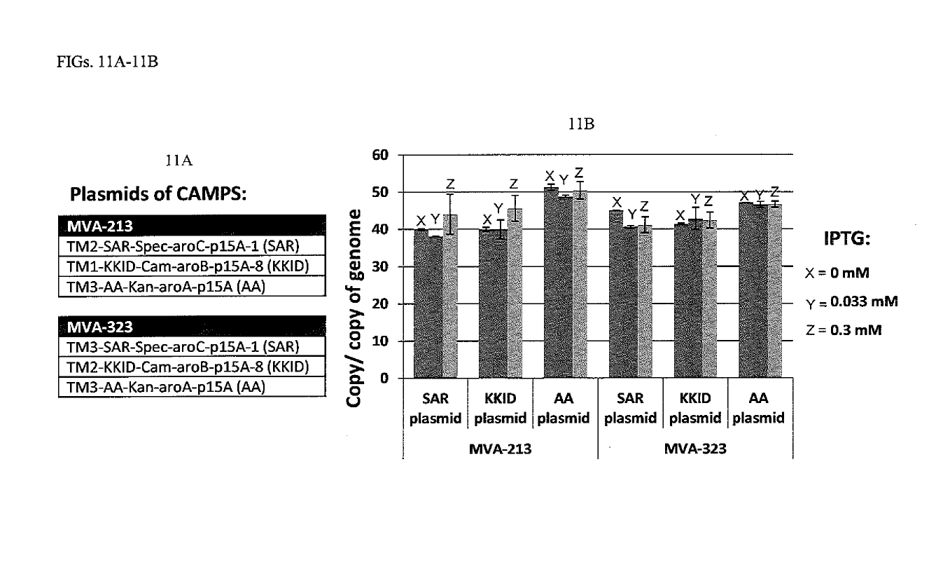

To overcome this, an antibiotic-free approach was proposed for the assembly of CAMPS (FIG. 10). As the rich medium contain essential metabolites for cell growth, all the CEG are non-essential when cultured with rich medium. By supplying all the specific metabolites used by CEG to the minimum medium, the cell should then be able to grow. The externally supplied metabolites can either be the direct product of the CEG or the intermediates in the linear metabolic pathway downstream of the CEG. With the supply of a combination of these key metabolites, a collection of modified minimum medium will allow the switch of CEG between its essential and non-essential statuses. By growing the strains with the multiple CEG knocked-out in the respective medium supplemented with the appropriate products iteratively, it should be possible to assemble strains harboring multiple plasmids with multiple CEG, which was performed as described below (FIG. 10).

Firstly, the growth properties of CEG knockout strains in various modified minimum mediums were tested. By supplying the direct product of CEG to the minimum medium with glucose as carbon source (MMG) (FIG. 8, the growth of engineered strains was rescued. Examples of this approach were the growth of MG1655 (DE3, ΔpdxH) strain when the minimal medium when supplemented with pyridoxal 5′-phosphate (PLP), the growth of MG1655 (DE3, ΔproC) strain when supplemented with proline (PRO), the growth of MG1655 (DE3, ΔpyrF) strain when supplemented with Uridine monophosphate (UMP) and the growth of MG 1655 (DE3, ΔargBCH) strain when supplemented with arginine (ARG) (Table 5). Furthermore, the engineered strains can also be rescued by supplying metabolites at several steps downstream of the medium CEG (FIG. 8) such as the growth of MG1655 (DE3, ΔaroB) strain when the medium was supplemented with shikimate (SK) and the growth of MG1655 (DE3, ΔargBCH) strain harboring T7-ADS-ispA-Cam-argH-p15AL2-11 plasmid when supplemented with ornithine (OR) (Table 5). The survival test validated that every chosen supplement here was efficiently utilized by the cell and complemented the lack of specific CEG (Table 5).

Next, the studies on the use of antibiotic-free procedures of introducing multiple CEG carrying plasmids were designed and tested (Table 6). For MG1655 (DE3, ΔaroABC) strain where all the selected CEG were in a linear pathway, the modified strains with T7-dxs-Kan-aroA-pET and T7-CYP450-CPR-Spec-aroC-pCL plasmids were firstly transformed into cells grown in the MMG+SK medium where aroB was non-essential. The cells were then further transformed with the T7-ADS-Cam-aroB-p15A plasmid and selected in MMG medium (Table 6, strain 1).

Similarly, MG1655 (DE3, AargBCH) strain was generated by introducing T7-ADS-ispA-Cam-argH-p15AL2-11 plasmid in the first step in cells grown in the MMG+OR medium. Then the T7-ADS-ispA-Cam-argB-p15AL2-10 and T7-ADS-ispA-Cam-argC-p15AL2-6 plasmids were introduced into cells grown in MMG medium (Table 6, strain 5). For quadruple CEG knockout strains: MG1655 (DE3, ΔaroABC, ΔproC) strain (Table 6, strain 2), MG1655 (DE3, ΔaroABC, ΔpdxH) strain (Table 6, strain 3), MG1655 (DE3, ΔaroABC, ΔpyrF) strain (Table 6, strain 4), three plasmids carrying aroA, aroB or aroC genes were first introduced using protocols similar to the assembly of MG1655 (DE3, ΔaroABC) strain (strain 1) while additional product (PRO, PLP or UMP) was supplied to the relevant mediums for each of the strain. Using MMG medium for selection, the last plasmid (T7-ADS-ispA-Cam-proC-p15AL2-4, T7-ADS-ispA-Cam-pdxH-p15AL2-9 or T7-ADS-ispA-Cam-pyrF-p15AL2-1) was then introduced into the corresponding strain in the third step. By combining the procedures of stain 1 and strain 5, the assembly protocol for sextuple CEG knockout strain: MG1655 (DE3, ΔaroABC, ΔargBCH) strain was similarly carried out (Table 6, strain 6) where ARG was supplied to make argB, argC and argH genes non-essential while transforming the aroA, aroB, aroC carrying plasmids. The results of these studies demonstrated the success in introducing multiple plasmids with a variety of CEG by using minimal medium supplemented with the appropriate chemicals which were products of the CEG activities.

TABLE 5
Survival test of mutant strains in modified minimum medium

| | Medium | | | | | | |
|---|---|---|---|---|---|---|---|
| | | MMG + | MMG + | MMG + | MMG + | MMG + | MMG + |
| Strain and plasmid | MMG | PLP | UMP | PRO | OR | ARG | SK |
| MG1655, DE3 | Yes | Yes | Yes | Yes | Yes | Yes | Yes |
| MG1655, DE3, ΔpdxH | No | Yes | No | No | N.A. | No | No |
| MG1655, DE3, ΔproC | No | No | No | Yes | N.A. | No | No |
| MG1655, DE3, ΔpyrF | No | No | Yes | No | N.A. | No | No |
| MG1655, DE3, ΔargBCH | No | No | No | No | N.A. | Yes | No |
| MG1655, DE3, ΔargBCH | No | No | No | No | Yes | Yes | No |
| T7-ADS-ispA-Cam-argH- | | | | | | | |
| p15AL2-11 | | | | | | | |
| MG1655, DE3, ΔaroB | No | No | No | Yes | No | No | No |
| MG1655, DE3, ΔaroABC | No | No | No | No | No | No | No |

Yes: Growth,
No: No growth.
N.A.: not aviable (the condition was not tested). The growth test was carried out in minimum medium with glucose as carbon source at 37° C. for 6 days with shaking (250 RPM).
MMG: minimum medium with glucose. Chemicals were supplied at 2 g/L inside the modified minimum medium.
Abbreviation for chemicals:
PLP: pyridoxal 5′-phosphate,
UMP: Uridine monophosphate,
SK: Shikimate,
PRO: Proline,
OR: Ornithine,
ARG: Arginine.

TABLE 6
Procedures to introduce multiple CEG carrying plasmids

| | Plasmid to transform | Step | Medium |
|---|---|---|---|
| Strain 1 | | | |
| MG1655, DE3, ΔaroABC | T7-dxs-Kan-aroA-pET | 1 | MMG + SK |
| | T7-CYP450-CPR-Spec-aroC-pCL | | |
| | T7-ADS-Cam-aroB-p15A | 2 | MMG |
| Strain 2 | | | |
| MG1655, DE3, ΔaroABC, | T7-dxs-Kan-aroA-pET | 1 | MMG + SK + |
| ΔproC | T7-CYP450-CPR-Spec-aroC-pCL | | PRO |
| | T7-ADS-Cam-aroB-p15A | 2 | MMG + PRO |
| | T7-ADS-ispA-Cam-proC-p15AL2-4 | 3 | MMG |
| Strain 3 | | | |
| MG1655, DE3, ΔaroABC, | T7-dxs-Kan-aroA-pET | 1 | MMG + SK + |
| ΔpdxH | T7-CYP450-CPR-Spec-aroC-pCL | | PLP |
| | T7-ADS-Cam-aroB-p15A | 2 | MMG + PLP |
| | T7-ADS-ispA-Cam-pdxH-p15AL2-9 | 3 | MMG |
| Strain 4 | | | |
| MG1655, DE3, ΔaroABC, | T7-dxs-Kan-aroA-pET | 1 | MMG + SK + |
| ΔpyrF | T7-CYP450-CPR-Spec-aroC-pCL | | UMP |
| | T7-ADS-Cam-aroB-p15A | 2 | MMG + UMP |
| | T7-ADS-ispA-Cam-pyrF-p15AL2-1 | 3 | MMG |
| Strain 5 | | | |
| MG1655, DE3, ΔargBCH | T7-ADS-ispA-Cam-argH-p15AL2-11 | 1 | MMG + OR |
| | T7-ADS-ispA-Cam-argB-p15AL2-10 | 2 | MMG |
| | T7-ADS-ispA-Cam-argC-p15AL2-6 | | |
| Strain 6 (proposed) | | | |
| MG1655, DE3, ΔaroABC, | T7-dxs-Kan-aroA-pET | 1 | MMG + SK + |
| ΔargBCH | T7-CYP450-CPR-Spec-aroC-pCL | | ARG |
| | T7-ADS-Cam-aroB-p15A | 2 | MMG + ARG |
| | T7-ADS-ispA-Cam-argH-p15AL2-11 | 3 | MMG + OR |
| | T7-ADS-ispA-Cam-argB-p15AL2-10 | 4 | MMG |
| | T7-ADS-ispA-Cam-argC-p15AL2-6 | | |

MMG: minimum medium with glucose.
Chemicals were supplied at 2 g/L inside the modified minimum medium.
Abbreviation for chemicals:
PLP: pyridoxal 5′-phosphate,
UMP: Uridine monophosphate,
SK: Shikimate,
PRO: Proline,
OR: Ornithine,
ARG: Arginine.
The construction procedures of strain 1-5 were experimentally validated and the construction procedure of strain 6 was proposed.

CAMPS for Metabolite Production

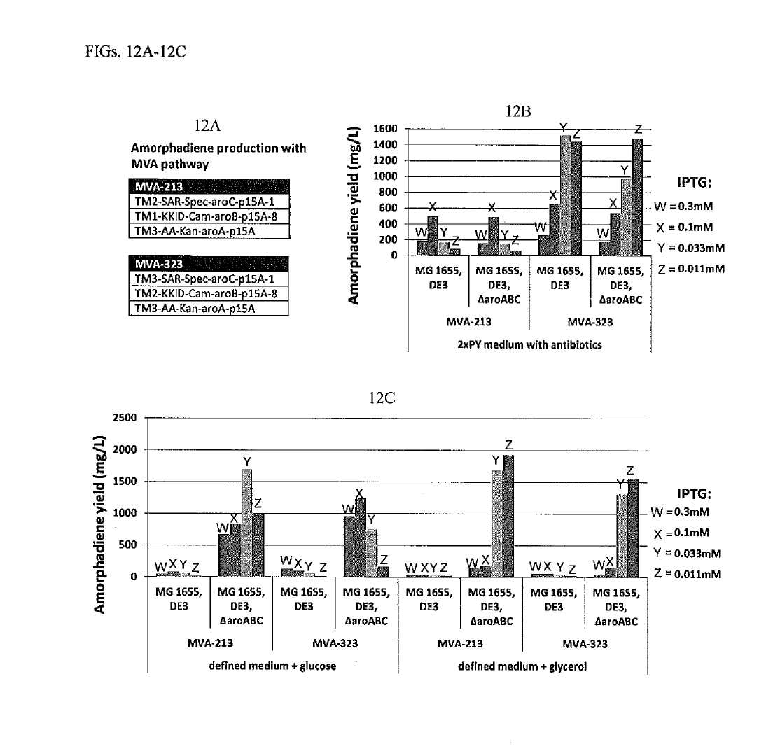

To demonstrate the utility of CAMPS, the production of amorphadiene through mevalonate (MVA) pathway was examined. The genes encoding the pathway enzymes were divided into three modules: the SAR module (hmgS, hmgR, atoB), the KKDI module (MVK, PMVK, MVD, idi) and the AA module (ADS, ispA). The modules were then separately placed into two sets of CAMPS plasmids: the MVA-213 set (TM2-SAR-Spec-aroC-p15A-1, TM1-KKID-Cam-aroB-p15A-8, TM3-AA-Kan-aroA-p15A) and the MVA-323 set (TM2-SAR-Spec-aroC-p15A-1, TM1-KKID-Cam-aroB-p15A-8, TM3-AA-Kan-aroA-p15A) where they were under the control of T7 promoter mutants of different strengths of controlling transcription (FIG. 12A). The combinations of mutant T7 promoters which enable high amorphadiene productivity were selected to drive the expression of pathway modules. The plasmids in each set were then transformed into the MG1655 (DE3, AaroABC) strain to assemble the CAMPS.

Firstly, plasmid stability was tested for these strains when cultured in antibiotic-free minimum medium with glucose as the carbon source (FIG. 12A). From the result, all the engineered plasmids in both sets had stable copy numbers in varying induction conditions which resulted in selection pressure not only because of the synthesis of the recombinant enzymes but also due to the perturbation of the global cellular metabolism. All the plasmids were found to be stably retained under different induction conditions, an essential feature in industrial scale fermentation process.

The production of amorphadiene were then compared when the two sets of plasmids were separately transformed into either the native strain (MG1655 (DE3)) or the engineered strain (MG1655 (DE3, ΔaroABC)). The strains were initially cultured in rich medium (2xPY++ medium) supplemented with three antibiotics (kanamycin, chloramphenicol and spectinomycin) to force the maintaining of all the plasmids. Although the MVA-213 set yielded less amorphadiene than the MVA-323 set as predicted, there was no difference between two strains with or without CAMPS (FIG. 12B) when the same set of plasmids were used. Those observations confirmed that the knockout strain with several essential gene disabled have similar properties in amorphadiene production as compared to the native strain.

Next, the productivity of the strains when cultured in antibiotic-free minimum medium with glucose or glycerol as the carbon source and various IPTG inductions were compared (FIG. 12C). The optimum yield of each strain with CAMPS was similar if not higher than when they were cultured in antibiotic conditions in rich medium. Clearly, the productivities using the native strain MG1655 (DE3) were significant lower at all conditions when compared to the strains engineered for CAMPS. The results proved that the CAMPS, in the absence of antibiotics, not only was able to retain the plasmid copy number but also maintained the productivity of metabolites.

Discussion

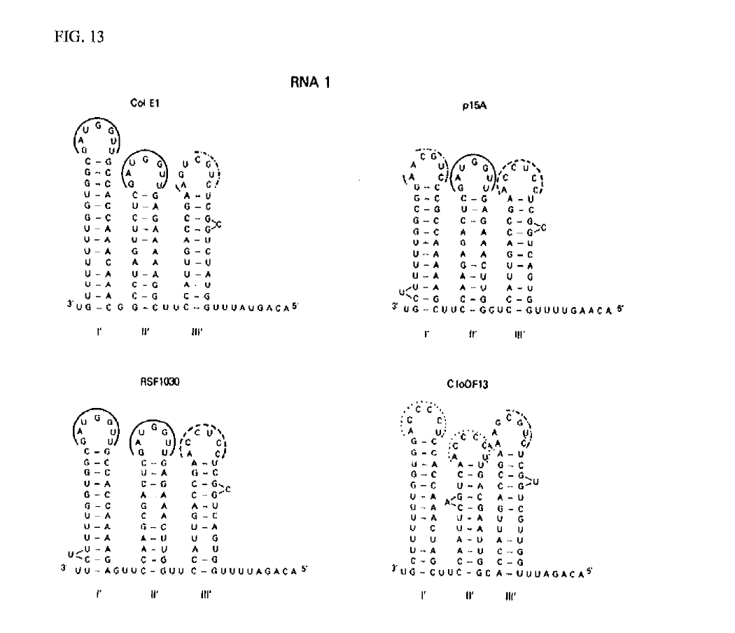

For metabolic engineering and synthetic biology, a stable multiple-plasmid system is needed. As compatibility is an important issue when using multiple of plasmids simultaneously, an option is to use plasmids from different compatibility groups which are limited in numbers and also highly variable in copy numbers. The mechanisms of plasmid replication were discovered decades ago. In p15A Ori, the regulation of plasmid copy number and the compatibility of different plasmids are high related. A key step in controlling plasmid copy number is the inhibition of plasmid replication by RNA I which recognizes a complementary stem-loop structure of RNA II—the RNA primer that initiates the plasmid replication, Based on the mechanism, various attempts had been made to engineer the copy number of a plasmid by manipulating the sequences in the stem-loop structures with good success while attention to the compatibility issue of these modified plasmids has yet to be determined. In nature, plasmids with slight different sequences in the stem-loop structures are known to be compatible and are thought to evolutionarily related. For example, the p15A like plasmids: p15A, Col E1, RSF 1030 and CloDF13 are compatible with each other to a certain degree (FIG. 13). Some other evidences have also suggested that the recognition of RNA I and RNA II accounts for the compatibility between plasmids.

As described herein, the loop sequences of RNA I/II were specifically engineered and the modified plasmids were proven to be compatible. Plasmids with loop sequences differing by as few as two bases were found to be compatible. From these observations, an unlimited number of compatible plasmids can be created. In addition, by engineering the sequences in the origin of replication, these plasmids have varying copy numbers when present in the host. To engineer metabolic pathways, other than selecting high copy number plasmids, plasmids with similar copy numbers to the parental plasmid (p15A Ori) can be selected for the ease of control. Another important differentiating factor in this study as compared to the use of different naturally compatible plasmids is that the modified plasmid library generated here share the same mechanisms of replication and resources.

To expand the plasmid library, compatible plasmids can be generated by engineering the 2nd as well as the 1st and 3rd loop of p15A series Ori. Other series of plasmid family with replication mechanisms controlled by the recognition between RNA loops can be also engineered similarly. An example is the IncIα plasmid family whereas Inc RNA regulates the repZ translation and Inc18 plasmid family whereby RNA III inhibits the transcriptional of RepR protein.

Another challenge of using multiple plasmids is the maintenance of the plasmids inside the host during large scale fermentation processes. The use of antibiotics is not only limited by the lack of different antibiotics available but also challenges the purification, regulatory and cost issues. There are several antibiotic-free systems for the maintenance of a single plasmid usually for the purpose of recombinant production. However, there is yet to be an example of a robust antibiotic-free multiple-plasmid system.

Described herein is the development of compositions and methods to construct a versatile antibiotic-free multiple-plasmid system in which the plasmids carried genes that complemented the auxotroph host thereby stably maintaining the plasmids even under conditions of cellular stress. The first challenge was to construct strains with multiple essential gene knockouts, which could not survive in the absence of the appropriate plasmids. To overcome this, the choice of the conditional essential genes (CEG) allowed the strains to be conveniently created in rich medium where those genes are conditionally non-essential. Nine CEG from various biosynthetic pathways were experimentally demonstrated separately or in combinations. It was shown that the plasmids were stable even when the cells were subject to high selection pressure. After strain construction, another challenge was the lack of an antibiotic-free approach to introduce multiple plasmids into the strain because the limitation in the efficiency of co-transformation of plasmids to E. coli. This issue was surmounted with the use of modified growth medium where the strains lacking certain CEG could be grown by supplementing with the appropriate products. As a result, all the plasmids can be stepwise transformed in an iterative manner using respective modified growth medium at each step.

With two features: compatible plasmids using the same replication mechanism and culture in antibiotic-free medium, this novel multi-plasmid system is beneficial to studies involving the simultaneous manipulation of large number of genes in industrial large scale production process. As a proof-of-concept, this was demonstrated by the production of amorphadiene where the native host yielded much less amorphadiene as compared to CAMPS strains in antibiotic free environment.

Methods

Chemical, Growth Medium and Bacteria Strain

Unless stated otherwise, all chemicals were purchased from either Sigma or Merck. The yeast extract and peptone were purchased from BD. The growth medium was prepared supplying 20 g/L glucose or glycerol as carbon source. The 2xPY medium contained: peptone (20 g/L), yeast extract (10 g/L) and NaCl (10 g/L). The 2xPY++ medium contained: peptone (20 g/L), yeast extract (10 g/L), NaCl (10 g/L), glucose (20 g/L), Tween 80 (0.5%), HEPES (50 mM) and was the rich medium used for amorphadiene production. X110-Gold (Stratagene) or DH5a (Invitrogen) strain was used for plasmid construction, Unless stated otherwise, all the cells were grown at 37° C. with shaking (250 rpm). In certain conditions, various kinds of antibiotics were supplied as following: ampicillin—100 mg/L, kanamycin—50 mg/L, chloramphenicol—34 mg/L, spectinomycin—100 mg/L. MG1655 (DE3) strain was the same as the one used in previous study [47] and was the strain used for studies involving the construction and characterization of novel compatible plasmids. Cell density (absorbance at 600 nm) was measured by SpectraMax 190 microplate reader.

Amorphadiene Production

For amorphadiene production experiment, 800 μL of cells were cultured together with 200 μL of dodecane phase and cultured at 28° C. with shaking (250 RPM) in 15 ml FalconTM tube. The experiments were carried out for two days for rich medium (2xPY++ medium) and for four days for growth medium (growth medium with glycerol or glucose). Amorphadiene was trapped in the dodecane phase and quantified as previously described [35]. The dodecane phase was diluted 100 times in ethyl acetate and the amorphadiene was quantified by Agilent 7890 gas chromatography/mass spectrometry (GC/MS) by scanning 189 and 204 m/z ions, using trans-caryophyllene as standard. The amorphadiene concentrations were adjusted to the volume of cell suspension (0.8 ml) for report.

Strain Construction

Based on the parental strain (MG1655 (DE3)), the knock-out strains were constructed using the method described in the paper [48]. The primer pairs KO-aroAF/KO-aroAR, KO-aroBF/KO-aroBR, KO-aroCF/KO-aroCR, KO-pdxHF/KO-pdxHR, KO-proCF/KO-proCR and KO-pyrFF/KO-pyrFR were used to knock out the aroA, aroB, aroC, pdxH, proC, and pyrF genes receptively. The KO-argBCHF/KO-argBCHR primer pairs were used to knockout the argBCH operon consisting of argB, argC and argH genes. The knock-out strains were confirmed by PCR analysis with the primers listed in section “Primers used to check the knockout strains”. The “in” primers were targeting at the sequences removed from the genome and “out” primers were targeting at the genome regions outside the removed sequences.

Plasmid Construction

The strains used in the study were listed in “Table 7”. The T7-ADS-ispA-Cam-p15A plasmid, T7-dxs-Kan-pET plasmid and T7-CYP450-CPR-Spec-pCL plasmid were from previous studies. All plasmid were constructed with CLIVA method and primers were listed in “Table 7”. The mutagenesis of 2nd loop of p15A Ori was carried by PCR amplification of T7-ADS-ispA-Cam-p15A plasmid with I-15ALoop2-F/I-15ALoop2-R degenerate primer pairs. The I-aroA-F/I-aroA-R, I-aroB-F/I-aroB-R and I-aroC-F/I-aroC-R primer pairs were used to amplify the aroA, aroB and aroC genes from the genome of MG1655 strain (ATCC) together with their RBS sequences. The genes were then inserted into plasmids at locations adjacent to the antibiotic resistant genes to form a polycistronic expression. The I-KAN(aroAr)f/I-KAN(aroAf)r, I-CAM(aroBr)f/I-CAM(aroBf)r and I-SPE(aroCr)f/I-SPE(aroCf)r primer pairs were used to amplify the vectors respectively.

TABLE 7
Plasmids used in the study

| | | | Antibiotic | |
|---|---|---|---|---|
| | Promoter and | Original of | resistant | |
| Name | gene | replication | gene | CEG |
| T7-ADS-ispA-Cam-p15A | T7-ADS-ispA | p15A | Cam | — |
| T7-dxs-Kan-p15A | T7-dxs | p15A | Kan | — |
| T7-ADS-ispA-Cam- | T7-ADS-ispA | p15AL2-5 | Cam | — |
| p15AL2-5 | | | | |
| T7-ADS-ispA-Cam- | T7-ADS-ispA | p15AL2-8 | Cam | — |
| p15AL2-8 | | | | |
| T7-ADS-ispA-Spec- | T7-ADS-ispA | p15AL2-8 | Spec | — |
| p15AL2-8 | | | | |
| T7-ADS-ispA-Spec-p15A | T7-ADS-ispA | p15A | Spec | — |
| T7-ADS-Cam-p15A | T7-ADS | p15A | Cam | — |
| T7-ADS-Cam-aroB-p15A | T7-ADS | p15A | Cam | aroB |
| T7-dxs-Kah-pET | T7-dxs | pBR322 | Kan | — |
| T7-dxs-Kan-aroA-pET | T7-dxs | pBR322 | Kan | aroA |
| T7-CYP450-CPR-Spec- | T7-CYP450- | RSF1010 | Spec | — |
| pCL | CPR | | | |
| T7-CYP450-CPR-Spec- | T7-CYP450- | RSF1010 | Spec | aroC |
| aroC-pCL | CPR | | | |
| T7-ADS-ispA-Cam-pdxH- | T7-ADS-ispA | p15AL2-9 | Cam | pdxH |
| p15AL2-9 | | | | |
| T7-ADS-ispA-Cam-proC- | T7-ADS-ispA | p15AL2-4 | Cam | proC |
| p15AL2-4 | | | | |
| T7-ADS-ispA-Cam-pyrF- | T7-ADS-ispA | p15AL2-1 | Cam | pyrF |
| p15AL2-1 | | | | |
| T7-ADS-ispA-Cam-argB- | T7-ADS-ispA | p15AL2-10 | Cam | argB |
| p15AL2-10 | | | | |
| T7-ADS-ispA-Cam-argC- | T7-ADS-ispA | p15AL2-6 | Cam | argC |
| p15AL2-6 | | | | |
| T7-ADS-ispA-Cam-argH- | T7-ADS-ispA | p15AL2-11 | Cam | argH |
| p15AL2-11 | | | | |
| TM3-AA-Kan-aroA-p15A | TM3-ADS- | p15A | Kan | argH |
| | ispA-Kan | | | |
| TM3-SAR-Spec-aroC- | TM3-hmgS- | p15AL2-1 | Spec | aroA |
| p15A-1 | hmgR-atoB | | | |
| TM2-SAR-Spec-aroC- | TM2-hmgS- | p15AL2-1 | Spec | aroC |
| p15A-1 | hmgR-atoB | | | |
| TM2-KKID-Cam-aroB- | TM2-MVK- | p15AL2-8 | Cam | aroB |
| p15A-8 | PMVK-MVD- | | | |
| | idi | | | |
| TM1-KKID-Cam-aroB- | TM1-MVK- | p15AL2-8 | Cam | aroB |
| p15A-8 | PMVK-MVD- | | | |
| | idi | | | |

Plasmid Copy Number Measurement

The copy number of plasmid was defined as the ratio of the copy of plasmid DNA and to the copy of the genomic DNA. The copy numbers were measured by quantitative PCR (qPCR) with a standard curve prepared using linearized plasmid DNA or PCR product (1-3 kb) containing the amplicon (80-120 bps). Typically, 5 μL of cells whose medium were removed by centrifugation were diluted in 100 μL of water. The mixture was then heated at 95° C. for 20 min to lyse the cells. The cell debris was then removed by mild centrifugation and the solution containing all the DNAs was dilute 20 times for qPCR. The qPCR reactions were carried out in 25 μl final volume containing 5 μl diluted DNA samples, 1× Xtensa Buffer (Bioworks), 200 nM of each primer, 2.5 mM MgCl2 and 0.75 U of iTaq DNA polymerase (iDNA). The reactions were analyzed using a BioRad iCycler 4™ Real-Time PCR Detection System (Bio-Rad) with SYBR Green I detection and the following protocol: an initial denaturation of 1 min at 95° C., followed by 40 cycles of 20 s at 95° C., 20 s at 60° C., and 20 s min at 72° C. A melt curve was then measured to check the melting temperature of the amplicon. For all the studies, technical duplicates or triplicates were carried out. Primers were listed in “Table 8-Primers used for plasmid copy number measurement”. The antibiotic resistant genes were measured to represent the amount of various plasmids with Cam-F/Cam-R, Kan-F/Kan-R and Spe-F/Spe-R primer pairs. For amorphadiene production study, the pathway genes were measured to represent the amount of various plasmids with hmgS-F/hmgS-R, MVK-F/MVK-R and ADS-F/ADS-R primer pairs. The cysG-F/cysG-R primer pair was used to measure the cysG gene which is one copy in the genome to represent the copy number of genomic DNA.

TABLE 8
Primers used in the study

| | |
|---|---|
| Name | Sequence |

Primers used for plasmid construction

| | |
|---|---|
| I-15ALoop2-F | CTCTTTGAAC*CGAGGTAACT*GGCTTG*GAGG |
| I-15ALoop2-R | CAAGCC*AGTTACCTCG*GTTCAAAGAG* |
| | TNNNNNGCTCAGAGAACCTTC |
| I-aroA-F | GGCGAGCC*AGCCTGTGG*GGTTT |
| I-aroA-R | GGCGAGGC*TATTTATTGCC*CGTTG |
| I-aroB-F | GGCGACACT*CGTCTGCGGG*TACAGTA |
| I-aroB-R | GGCGTTACG*CTGATTGACA*ATCGG |
| I-aroC-F | GGCGACACG*CAAACACAAC*AATAACGG |
| I-aroC-R | GGCGTTACC*AGCGTGGAAT*ATCAGTC |
| I-pdxH-F | AGTTATT*GGTGCCCTTA* |
| | CCCACTGACAATCCGTAAAGA |
| I-pdxH-R | TAGCACC*AGGCGTT* |
| | TCAGGGTGCAAGACGATCAA |
| I-proC-F | GTTATT*GGTGCCCTTA* |
| | GCCAGCGATTATCAAAACAA |
| I-proC-R | TAGCACC*AGGCGTT*CGGCGAAAGTCATCAGGA |
| I-pyrF-F | AGTTATT*GGTGCCCTTA* |
| | GGTGCCCATCATCAAGAAGG |
| I-pyrF-R | TAGCACC*AGGCGTT* |
| | CGGCTGTTGGAATCACTCATC |
| I-argB-F | AGTTATT*GGTGCCCTTA* |
| | GCGGAAACGCAGTCTCTTA |
| I-argB-R | TAGCACC*AGGCGTT* |
| | TGAAATTCAATGCCGGAAAG |
| I-argC-F | AGTTATT*GGTGCCCTTA* |
| | ACCAGACATAAGAAGGTGAATAGC |
| I-argC-R | TAGCACC*AGGCGTT* |
| | ACCCTTAAATAAGAGACTGCGTT |
| I-argH-F | AGTTATT*GGTGCCCTTA* |
| | GGCATTGAATTTCAAAATAAGG |
| I-argH-R | TAGCACC*AGGCGTT*AAAAGCCCGGCGATAAG |
| I-KAN(aroAf)r | CCACAGGC*TGGCTCGCC* |
| | AGAGTCCCGCTCAGAAGAA |
| I-KAN(aroAr)f | GGCAATAA*ATAGCCTCGCC* |
| | TTCGAAATGACCGACCAA |
| I-KAN(aroBf)r | CCCGCAGAC*GAGTGTCGCC* |
| | TAAGGGCACCAATAACTGCC |
| I-KAN(aroBr)f | TGTCAATCA*GCGTAACGCC* |
| | AACGCCTGGTGCTACGC |
| I-SPE(aroCr)f | ATTCCACGC*TGGTAACGCC* |
| | TTCACCGACAAACAACAGATAA |
| I-SPE(aroCf)r | GTTGTGTTT*GCGTGTCGCC* |
| | GTGCTTAGTGCATCTAACGCT |

Primers used for plasmid copy number measurement

| | |
|---|---|
| Cam-F | CTGGAGTGAATACCACGACG |
| Cam-R | GGATTGGCTGAGACGAAAA |
| Kan-F | GTCCGGTGCCCTGAATGAA |
| Kan-R | CCCAATAGCAGCCAGTCCCT |
| Spe-F | CGCTCACGCAACTGGTCCAGAA |
| Spe-R | CGAGGCATAGACTGTACCCCAAA |
| cysG-F | TTGTCGGCGGTGGTGATGTC |
| cysG-R | ATGCGGTGAACTGTGGAATAAACG |
| hmgS-F | GGTAGAGACGCCATTGTAG |
| hmgS-R | CCGATCCACATAGCAACA |
| ADS-F | CCGTATCGTAGAATGCTATT |
| ADS-R | CCGCTTTGGTGAAGAATA |
| MVK-F | GCGTTGAGAACCTACCTGCTAAT |
| MVK-R | ATCCTCGGTGATGGCATTGAA |

Primers used for knockout strain construction

| | |
|---|---|
| KO-aroAF | GTTGTAGAGAGTTGAGTTCATGGAATCCCTGACG |
| | TTACAACCCATCGCTCGTGTAGGCTGGAGCTGCT |
| | TC |
| KO-aroAR | CATTCAGGCTGCCTGGCTAATCCGCGCCAGCTGC |
| | TCGAAATAATCCGGAGCATATGAATATCCTCCTT |
| | AG |
| KO-aroBF | CTGCGGGTACAGTAATTAAGGTGGATGTCGCGTT |
| | ATGGAGAGGATTGTCGTGTAGGCTGGAGCTGCTT |
| | C |
| KO-aroBR | CCCCATTTCAGCTTCAATGGCATGACCAAAGGTG |
| | TGTCCCAGATTCAGAGCATATGAATATCCTCCTT |
| | AG |
| KO-aroCF | CGGAGCCGTGATGGCTGGAAACACAATTGGACAA |
| | CTCTTTCGCGTAACCGTGTAGGCTGGAGCTGCTT |
| | C |
| KO-aroCR | CCAGCGTGGAATATCAGTCTTCACATCGGCATTT |
| | TGCGCCCGTTGCCGAGCATATGAATATCCTCCTT |
| | AG |
| KO-pdxHF | AAACGCGACCGCATCGTCTTGAATAACTGTCAGT |
| | TACAAAATCCACAGCGTGTAGGCTGGAGCTGCTT |
| | C |
| KO-pdxHR | TCAGGGTGCAAGACGATCAATCTTCCACGCATCA |
| | TTTTCACGCTGGTACGAGCATATGAATATCCTCC |
| | TTAG |
| KO-proCF | TCTATTGTGTCGCGCTTTTGCCTTCCGGCATAGT |
| | TCTGTTTATGCTTCTGTGTAGGCTGGAGCTGCTT |
| | C |
| KO-proCR | CATACACTTCGTCATCGCTTCGATCACTGCAGCA |
| | CGGAAGCCTTTCTCTTGAGCATATGAATATCCTC |
| | CTTAG |
| KO-pyrFF | TAGAATGCTCGCCGTTTACCTGTTTCGCGCCACT |
| | TCCGGTGCCCATCATCGTGTAGGCTGGAGCTGCT |
| | TC |
| KO-pyrFR | GTTACCGGGCGACCAATCACCATATAATCAACAC |
| | CAGCCGACAACGCCTGAGCATATGAATATCCTCC |
| | TTAG |
| KO-argBCHF | GCCGTAAGGTGAATGTTTTACGTTTAACCTGGCA |
| | ACCAGACATAAGAAGGTGTAGGCTGGAGCTGCTT |
| | C |
| KO-argBCHR | CCCTAACCGAGCCTGCGCAAAAGCAATCGCCTGC |
| | GCCACCTGCTGCGGTGAGCATATGAATATCCTCC |
| | TTAG |

Primers used to check the knockout strains

| | |
|---|---|
| aroA-out-F | CGCTGACAGACTTCATGGTTGAG |
| aroA-in-R | AGGGCACCTTCTGCGTGTAATG |
| aroA-out-R | CGGCACAGCCCTGATTGG |
| aroB-out-F | AACGCAATCCGCTGTATGAAGA |
| aroB-in-R | CAGAGGAGCCAGGGTTTCGTT |
| aroB-out-R | CCGCTGCGAACTTCACTCTTACC |
| aroC-out-F | TAACGGCGGCGATGGTGT |
| aroC-in-R | ACAAGCCAATGCTGGTGCCG |
| aroC-out-R | CCTGGCTACTCAGACGCTGGATAA |
| pdxH-out-F | ACGGAATCTATGTTTTCTGGTCG |
| pdxH-in-R | CTTTTTCGTCGTAATGTTTGAGTA |
| proC-out-F | CATCCACCCAAATTGTCATAAA |
| proC-in-R | CGATTCTGCGGCGTTGAT |
| pyrF-out-F | CTGCCAGGGGAGAAATCG |
| pyrF-in-R | GAACTCCTGACCGAATACCTGT |
| argBCH-out-F | GTTTTTCATTGTTGACACACCTC |
| argBCH-in-R | ACCCTTAAATAAGAGACTGCGTT |

(*the positions with thiophosphate modification)

Articles such as “a”, “an”, “the” and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

Embodiments in which any one or more limitations, elements, clauses, descriptive terms, etc., of any claim (or relevant description from elsewhere in the specification) is introduced into another claim are provided. For example, a claim that is dependent on another claim may be modified to include one or more elements or limitations found in any other claim that is dependent on the same base claim. It is expressly contemplated that any amendment to a genus or generic claim may be applied to any species of the genus or any species claim that incorporates or depends on the generic claim.

Where a claim recites a composition, methods of using the composition as disclosed herein are provided, and methods of making the composition according to any of the methods of making disclosed herein are provided. Where a claim recites a method, a composition for performing the method is provided. Where elements are presented as lists or groups, each subgroup is also disclosed. It should also be understood that, in general, where embodiments or aspects is/are referred to herein as comprising particular element(s), feature(s), agent(s), substance(s), step(s), etc., (or combinations thereof), certain embodiments or aspects may consist of, or consist essentially of, such element(s), feature(s), agent(s), substance(s), step(s), etc. (or combinations thereof). It should also be understood that, unless clearly indicated to the contrary, in any methods claimed herein that include more than one step or act, the order of the steps or acts of the method is not necessarily limited to the order in which the steps or acts of the method are recited.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1.-107. (canceled)

108. A method of expressing two or more nucleic acid sequences, comprising:
a) introducing into a host cell two or more plasmids, wherein (i) the host cell lacks one or more conditional essential genes and (ii) the two or more plasmids comprise the two or more nucleic acid sequences and the one or more conditional essential genes, and each plasmid comprises an origin of replication (Ori) that is identical except for one or more loop sequences in the Ori of each plasmid; and
b) maintaining the host cell under conditions in which the two or more nucleic acid sequences and the one or more conditional essential genes are expressed in the host cell, thereby expressing the two or more nucleic acid sequences.

109. The method of claim 108, wherein the host cell is an auxotroph host cell.

110. The method of claim 108, wherein the host cell is a prokaryotic host cell.

111. The method of claim 110, wherein the prokaryotic host cell is E. coli.

112. The method of claim 108, wherein the one or more conditional essential genes are essential when the host cell is cultured in a selection culture media but not in a non-selection culture, wherein the selection culture media comprises one or more sugars.

113. The method of claim 112, wherein the one or more sugars is glucose, glycerol or a combination thereof.

114. The method of claim 112, further comprising adding one or more chemicals or metabolites produced by one or more polypeptides or intermediates thereof to the selection culture media, wherein the one or more conditional essential genes encode the one or more polypeptides in one or more biochemical pathways of the host cell.

115. The method of claim 114, wherein the one or more intermediates are downstream in a biochemical pathway of the one or more CEGs.

116. The method of claim 115, wherein the one or more intermediates comprise pyridoxal 5′-phosphate (PLP), proline (PRO), uridine monophosphate (UMP), arginine (ARG), shikimate (SK), ornithine (OR) or a combination thereof.

117. The method of claim 112, wherein the biochemical pathway is a metabolic pathway of the host cell, wherein the one or more conditional essential genes encode one or more metabolic enzymes of the host cell.

118. The method of claim 117, wherein the one or more metabolic enzymes comprise aroA, aroB, aroC, pdxH, pyrF, proC, argB, arC, argH or a combination thereof.

119. The method of claim 108, wherein each conditional essential gene is inserted into a separate plasmid.

120. The method of claim 108, wherein at least one plasmid comprises a recognition site for RNase H.

121. The method of claim 108, wherein the one or more loop sequences in the Ori of each plasmid differs by one or more nucleotides.

122. The method of claim 121, wherein the Ori of each plasmid comprises 3 loops and one or more nucleotides in a second loop of the Ori of each plasmid differs.

123. The method of claim 108, wherein the two or more nucleic acid sequences are expressed under antibiotic-free conditions.

124. The method of claim 108, wherein the two or more nucleic acid sequence is one or more genes.

125. The method of claim 124, wherein the one or more genes are overexpressed by the host cell.

126. A method of expressing two or more nucleic acid sequences, comprising:
maintaining a host cell comprising two or more plasmids wherein
a) the host cell lacks one or more conditional essential genes;
b) the two or more plasmids comprise the two or more nucleic acid sequences and the one or more conditional essential genes, and each plasmid comprises an origin of replication (Ori) that is identical except for one or more loop sequences in the Ori of each plasmid; and
c) under conditions in which the two or more nucleic acid sequences and the one or more conditional essential genes are expressed in the host cell, thereby expressing the two or more nucleic acid sequences.

127. A method of preparing a library of compatible plasmids, comprising
a) introducing into a host cell two or more plasmids, wherein (i) the host cell lacks one or more conditional essential genes and (ii) the two or more plasmids comprise two or more nucleic acid sequences and the one or more conditional essential genes, and each plasmid comprises an origin of replication (Ori) that is identical except for one or more loop sequences in the Ori of each plasmid; and
b) maintaining the host cell under conditions in which the two or more nucleic acid sequences and the one or more conditional essential genes are expressed in the host cell, thereby preparing a library of compatible plasmids.
